(19) United States
(12) Patent Application Publication
(10) Pub. No.: US 2025/0041397 A1
(43) Pub. Date: Feb. 6, 2025

(54) EXPRESSION OF BORRELIA BURGDORFERI OUTER SURFACE PROTEIN A IN PLANTS AND PLANT PRODUCED VACCINE FOR SAME

(71) Applicant: Applied Biotechnology Institute, Inc., San Luis Obispo, CA (US)

(72) Inventors: John Howard, San Luis Obispo, CA (US); Erin Egelkrout, San Luis Obispo, CA (US)

(21) Appl. No.: 18/678,214

(22) Filed: May 30, 2024

Related U.S. Application Data
(60) Provisional application No. 63/516,625, filed on Jul. 31, 2023.

(51) Int. Cl. A61K 39/02; A61K 36/899; A61K 39/00; C12N 15/82

(52) U.S. Cl. CPC A61K 39/0225; A61K 36/899; C12N 15/8216; A61K 2039/542; A61K 2039/552; A61K 2039/575

(57) ABSTRACT

Vaccines and methods of expressing a polypeptide of Borrelia burgdorferi are provided in which a protective response to Borrelia burgdorferi is produced when administered to an animal. The vaccine provides for expression of the Borrelia burgdorferi OspA polypeptide in a plant or plant part, linked to a promoter preferentially directing expression to embryo tissue of the plant or plant part. Further embodiments provide that the polypeptide may be targeted to the apoplast/cell wall or the endoplasmic reticulum. Increased expression levels in the plant or plant part are obtained. The plant or plant materials in an embodiment may be orally administered.

### CROSS-REFERENCE TO RELATED APPLICATION

This application claims priority under 35 U.S.C. § 119 to Provisional Application U.S. Ser. No. 63/516,625, filed on Jul. 31, 2023, which is herein incorporated by reference in its entirety including without limitation, the specification, claims, and abstract, as well as any figures, tables, or examples thereof.

### GRANT REFERENCE

This invention was made with government support under Grant R43AI152650 awarded by the National Institutes of Health/National Institute of Allergy and Infectious Diseases. The Government has certain rights in the invention.

### SEQUENCE LISTING XML

The instant application contains a sequence listing, which has been submitted in XML file format by electronic submission and is hereby incorporated by reference in its entirety. The XML file, created on May 27, 2024, is named P14502US01.xml and is 23,765 bytes in size.

### BACKGROUND

Lyme disease is the most prevalent vector borne disease in North America and Europe. It is a major public health concern throughout the US Northeast, Mid-Atlantic and Midwest regions where the disease is endemic and its range is expanding. The infectious agent, the spirochete Borrelia burgdorferi sensu lato (Borreliella genus novum under consideration) is transmitted to vertebrate hosts by Ixodes ticks. Humans are incidental hosts of B. burgdorferi. If diagnosed early, Lyme disease can be successfully treated with antibiotics; late Lyme disease involves a substantial convalescent period and can cause irreparable harm. Currently, there is no vaccine against Lyme disease available for human use although a second generation OspA vaccine, VLA15, is undergoing Phase III clinical trials. Thus, a sure way to avoid this debilitating disease is to avoid ticks. This is obviously impossible for those who work or entertain themselves outside in endemic areas. Independently of how successful human vaccination may or may not be in terms of efficacy and compliance rates, there is continued interest by the community in development of strategies to break the enzootic cycle of B. burgdorferi given that a lower burden of this spirochete in the environment should translate into a lower risk of exposure to infected ticks. Three reservoir targeted vaccines (RTV) based on OspA have been developed by different labs. However, Dr. Gomes-Solecki developed the only RTV that is undergoing testing for USDA license approval to be administered to Peromyscus leucopus. This RTV reduced nymphal infection prevalence up to 76% in a 5-year field trial and is expected to reduce the burden of B. burgdorferi in the environment. An alternative to the E. coli delivered OspA that would allow for mass production of large amounts of antigen in a stable, efficacious and easy to use form, at a low cost, would also be welcomed by commercial partners as it may be more amenable to broadcast delivery in non-peridomestic remote locations where issues of feeding wildlife are not pertinent.

### SUMMARY

A vaccine for Borrelia burgdorferi is provided that is produced from a plant. Provided are methods of producing a protective response to Borrelia burgdorferi in an animal. When the plant or plant product is orally administered to an animal, a protective response is observed. Embodiments provide orally administering a plant or plant product including the OspA polypeptide of Borrelia burgdorferi. Embodiments provide a promoter preferentially directing expression to seed of a plant. Further embodiments provide for targeting expression of the OspA polypeptide of Borrelia burgdorferi to the apoplast or endoplasmic reticulum. Additional embodiments provide the construct and plasmids for expression of the same at high levels in plants. Embodiments provide expression levels of at least 10 mg/kg in seed of the plant.

While multiple embodiments are disclosed, still other embodiments of the present disclosure will become apparent based on the detailed description, which shows and describes illustrative embodiments of the disclosure. Accordingly, the figures and detailed description are to be regarded as illustrative in nature and not restrictive.

### BRIEF DESCRIPTION OF THE DRAWINGS

The following drawings form part of the specification and are included to further demonstrate certain embodiments. In some instances, embodiments can be best understood by referring to the accompanying figures in combination with the detailed description presented herein. The description and accompanying figures may highlight a certain specific example, or a certain embodiment. However, one skilled in the art will understand that portions of the example or embodiment may be used in combination with other examples or embodiments.

FIG. 1 shows diagrams of the expression cassettes. All have embryo preferred promoters (pr44, SEQ ID NO: 11). LDA and LDC contain the native OspA signal sequence and LDB and LDD contain the barley alpha amylase signal sequence. LDC and LDD contain the KDEL signal sequence for targeting to the ER.

FIG. 2 shows western blots. Pooled seed (10 each) from individual plants representing independent insertion events for all four constructs were pulverized and extracted in 1×PBS+0.05% Tween. Untransformed Hill maize seed is a negative control and E. coli produced OspA is added as a positive control. Extracts were loaded on an SDS PAGE gel under reducing conditions. The blot was transferred to membrane and incubated overnight with mouse mAb 184 to OspA at 1:1000. The blots were developed using anti-mouse IgG conjugated to alkaline phosphatase and BCIP/NBT.

FIGS. 3A-D shows the OspA plasmid maps. FIG. 3A shows LDA (9317), FIG. 3B shows LDB (9318), FIG. 3C shows LDC (9319), and FIG. 3D shows LDD (9320).

### DETAILED DESCRIPTION

The proteins presented on the outer surface of B. burgdorferi switch as the spirochete transits between the tick vector and the mammalian host. In unfed ticks, B. burgdorferi produces outer surface protein A (OspA) while it resides in the tick midgut. As the tick engorges with blood during feeding, B. burgdorferi migrates from the midgut to the salivary gland where it downregulates expression of OspA and upregulates OspC to mediate its upcoming infection of mammals. Thus, when a nymphal tick feeds on mammals vaccinated with OspA (including humans), live spirochetes are killed in the tick midgut because that is where B. burgdorferi presents most of its OspA in the surface. As such, B. burgdorferi are no longer available to migrate to the salivary gland to be transmitted to the next host. Furthermore, OspA antibodies also reduce acquisition of Bb by uninfected larval ticks feeding on infected mice. Hence, OspA is the ideal target for development of transmission-blocking vaccines to prevent B. burgdorferi infection. Effective vaccines based on recombinant OspA have been developed for humans, dogs, and mice. OspC has been long considered a vaccine candidate against Lyme disease. However, OspC is not expressed by B. burgdorferi in the tick midgut but instead it is expressed in the salivary gland to enable infection of the host. Thus, OspC is not an antigen expected to produce an effective transmission blocking vaccine. It could however produce an effective canonical vaccine to afford protection of the host if proven effective after challenge with naturally infected ticks. Due to its diversity multiple attempts have produced tetravalent, octavalent and more recently, chimerotope vaccinogens that look promising. However, efficacy against challenge with ticks carrying multiple strains of B. burgdorferi has not yet been achieved.

To develop alternative vaccination strategies, several groups have explored oral delivery of subunit vaccines as they cannot replicate and are inherently safe. A concern regarding efficacy of oral delivery of subunit vaccines is the vulnerability to digestion of the antigen in the gastrointestinal tract before it can induce an immune response. Purified proteins rapidly degrade in the digestive tract and require much higher antigen doses (100-1000-fold) than those typically given by injection, adding significantly to the cost. Microencapsulation can protect the antigen and lower the amount of antigen required but this also adds cost.

One approach to producing high concentrations of subunit vaccines that will be protected against mucosal digestion at or below the current cost of parenterally delivered vaccines is to express the immunogen candidate in a plant host that will allow post-translational modifications in a eukaryotic cell. Plant-based systems can supply kilogram quantities of recombinant proteins, including vaccine antigens, with very low capital requirements for equipment and overhead costs compared to other expression systems. Furthermore, when edible tissue is used, the need for purification can be eliminated, which accounts for the vast majority of cost in producing a recombinant protein. With the appropriate choice of tissue for expression, processed material may be stable at ambient temperatures for years. This potential has led to the development of an array of plant-based vaccine candidates that elicit an immune response, that confer protection against viral challenge in animal studies and have demonstrated safety and efficacy in human clinical trials with E. coli LT-B.

Despite showing promise, with the exception of the recent regulatory approval in Canada of a parenterally delivered Covid-19 vaccine produced in tobacco, no plant-produced vaccines have been commercialized. One problem has been the low levels of antigen accumulation in the host resulting in the need to ingest an impractical amount of material to provide an acceptable immune response. Other hurdles have included poor stability and inactivation of the antigen on processing, and difficulty in scaling-up. Therefore, while there is ample demonstration that an orally delivered, plant-produced subunit vaccine can work, practical hurdles have limited implementation.

The present disclosure applies a technology using expression in maize grain that can overcome these barriers to viable plant-produced vaccines to develop a new system for low-cost production of a proven oral transmission blocking vaccine against a bacterium, B. burgdorferi. There is precedent for potential development of a vaccine against Bb in plants. OspA has been expressed in tobacco. Lipidation of OspA, which is important but not essential for immunogenicity has been demonstrated in tobacco. However, tobacco is not easily stored at ambient temperatures; it must be processed immediately after harvest, and after extraction, the protein must also be purified immediately to prevent degradation. Another plant-based system tested was rice. OspA expressed in rice elicited an immune response in mice, but the vaccine was not fully developed. Nonetheless, these examples provide evidence that development of OspA subunit vaccines in plants is feasible. This vaccine can be applicable to human, companion animal, and reservoir targeted use, with a focus on reservoir targeted use.

In an embodiment, maize grain is used as a basis for the production of the Borrelia burgdorferi OspA subunit vaccine. High expression levels of at least 10 mg/kg of whole seed are obtained. An embodiment provides for a range of about 10 mg/kg to 200 mg/kg. Further embodiments provide for expression at least 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 25 mg/kg, 30 mg/kg, 35 mg/kg, 40 mg/kg, 50 mg/kg, 60 mg/kg, 70 mg/kg, 80 mg/kg, 90 mg/kg, 100 mg/kg, 150 mg/kg, 200 mg/kg of whole seed or more or amounts in-between.

Oral administration of the plant, plant part or a product produced from the plant part, such as a seed, grain, flour or other edible composition comprising the plant, plant part or product produced therefrom comprising Borrelia burgdorferi OspA protein can result in protection against challenge for the subject animal. The subject animal may or may not produce detectable antibodies in response, but the animal will have decreased morbidity or mortality resulting from administration of the vaccine, such that upon exposure to disease challenge, the animal is able to combat the infection. The compositions of the disclosure may also induce a serum response as well as a mucosal response. The serum response in an embodiment is within the range of two to 100-fold more than the control. In another embodiment the response can be 5 times, 10 times, 15 times, 20 times, 25 times, 30 times, 35 times, 40 times, 45 times, 50 times, 55 times, 60 times, 65 times, 70 times, 75 times, 80 times, 85 times, 90 times, 95 times or more greater than control animals not receiving vaccination, or amounts in-between.

As used herein, the term “animal” or “subject” or “subject animal” is intended to include human beings.

As used herein, the terms nucleic acid or polynucleotide refer to deoxyribonucleotides or ribonucleotides and polymers thereof in either single- or double-stranded form. The sequence used to make the vaccine may be obtained from any source, such as a biological source in isolating from a biological sample or can refer to a sequence synthetically produced based upon the sequence obtained from the sample. As such, the terms include RNA and DNA, which can be a gene or a portion thereof, a cDNA, a synthetic polydeoxyribonucleic acid sequence, or the like, and can be single-stranded or double-stranded, as well as a DNA/RNA hybrid. Furthermore, the terms are used herein to include naturally-occurring nucleic acid molecules, which can be isolated from a cell, as well as synthetic molecules, which can be prepared, for example, by methods of chemical synthesis or by enzymatic methods such as by the polymerase chain reaction (PCR). Unless specifically limited, the terms encompass nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Cassol et al. (1992); Rossolini et al. (1994) Mol. Cell. Probes 8:91-98). The term nucleic acid is used interchangeably with gene, cDNA, and mRNA encoded by a gene.

Nucleic acids employed here include those that encode an entire polypeptide as well as those that encode a subsequence of the polypeptide or produce a fragment that provides a protective response. For example, nucleic acids that encode a polypeptide which is not full-length but nonetheless has protective activity against Borrelia burgdorferi. The disclosure includes not only nucleic acids that include the nucleotide sequences as set forth herein, but also nucleic acids that are substantially identical to, correspond to, or substantially complementary to, the exemplified embodiments. For example, the disclosure includes nucleic acids that include a nucleotide sequence that is at least about 70% identical to one that is set forth herein, more preferably at least 75%, still more preferably at least 80%, more preferably at least 85%, 85.5% 86%, 86.5% 87%, 87.5% 88%, 88.5%, 89%, 89.5% still more preferably at least 90%, 90.5%, 91%, 91.5% 92%, 92.5%, 93%, 94.5%, 94%, 94.5% and even more preferably at least about 95%, 95.5%, 96%, 96.5%, 97%, 97.5%, 98%, 98.5%, 99%, 95.5%, 100% identical (or any percentage in between) to an exemplified nucleotide sequence. The nucleotide sequence may be modified as described previously, so long as any polypeptide produced is capable of inducing the generation of a protective response.

The nucleic acids can be obtained using methods that are known to those of skill in the art. Suitable nucleic acids (e.g., cDNA, genomic, or subsequences) can be cloned, or amplified by in vitro methods such as the polymerase chain reaction (PCR) using suitable primers, the ligase chain reaction (LCR), the transcription-based amplification system (TAS), or the self-sustained sequence replication system (SSR). A wide variety of cloning and in vitro amplification methodologies are well-known to persons of skill in the art. Examples of these techniques and instructions sufficient to direct persons of skill through many cloning exercises are found in Berger and Kimmel, Guide to Molecular Cloning Techniques, Methods in Enzymology 152 Academic Press, Inc., San Diego, Calif. (Berger); Sambrook et al. (2001) Molecular Cloning—A Laboratory Manual (Third ed.) Vol. 1-3, Cold Spring Harbor Laboratory, Cold Spring Harbor Press, NY, (Sambrook et al.); Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Cashion et al., U.S. Pat. No. 5,017,478; and Carr, European Patent No. 0,246,864. Examples of techniques sufficient to direct persons of skill through in vitro amplification methods are found in Berger, Sambrook, and Ausubel, as well as Mullis et al., (1987) U.S. Pat. No. 4,683,202; PCR Protocols A Guide to Methods and Applications (Innis et al., eds) Academic Press Inc. San Diego, Calif. (1990) (Innis); Amheim & Levinson (Oct. 1, 1990) C& EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87, 1874; Lomell et al. (1989) J. Clin. Chem., 35: 1826; Landegren et al., (1988) Science 241: 1077-1080; Van Brunt (1990) Biotechnology 8: 291-294; Wu and Wallace (1989) Gene 4: 560; and Barringer et al. (1990) Gene 89: 117. Improved methods of cloning in vitro amplified nucleic acids are described in Wallace et al., U.S. Pat. No. 5,426,039. Nucleic acids or subsequences of these nucleic acids, can be prepared by any suitable method as described above, including, for example, cloning and restriction of appropriate sequences.

“Codon optimization” can be used to optimize sequences for expression in an organism and is defined as modifying a nucleic acid sequence for enhanced expression in the cells of the organism of interest, e.g., maize, by replacing at least one, more than one, or a significant number, of codons of the native sequence with codons that are more frequently or most frequently used in the genes of that organism. Various species exhibit particular bias for certain codons of a particular amino acid.

As used herein, a “polypeptide” refers generally to peptides and proteins. In certain embodiments the polypeptide may be at least two, three, four, five, six, seven, eight, nine or ten or more amino acids or more or any amount in-between. A peptide is generally considered to be more than fifty amino acids. The terms “fragment,” “derivative” and “homologue” when referring to the polypeptides according to the present disclosure, means a polypeptide which retains essentially the same biological function or activity as the polypeptide, that is, act as an antigen and/or provide treatment for and/or protection against disease. Such fragments, derivatives and homologues can be chosen based on the ability to retain one or more of the biological activities of the polypeptide, that is, act as an antigen and/or provide treatment for and/or protection against the pathogen. The polypeptide vaccines of the present disclosure may be recombinant polypeptides, natural polypeptides or synthetic polypeptides, preferably recombinant polypeptides. One skilled in the art appreciates that it is possible that the protective polypeptide may be expressed by the gene in the host cells and the plant composition administered to the animal or extracted from the plant prior to administration.

“Conservatively modified variants” applies to both amino acid and nucleic acid sequences. With respect to particular nucleic acid sequences, conservatively modified variants refer to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent substitutions” or “silent variations,” which are one species of “conservatively modified variations.” Every polynucleotide sequence described herein which encodes a polypeptide also describes every possible silent variation, except where otherwise noted. Thus, silent substitutions are an implied feature of every nucleic acid sequence which encodes an amino acid. One of skill will recognize that each codon in a nucleic acid (except AUG, which is ordinarily the only codon for methionine) can be modified to yield a functionally identical molecule by standard techniques. In some embodiments, the nucleotide sequences that encode a protective polypeptide are preferably optimized for expression in a particular host cell (e.g., yeast, mammalian, plant, fungal, and the like) used to produce the polypeptide or RNA.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” referred to herein as a “variant” where the alteration results in the substitution of an amino acid with a chemically similar amino acid. Conservative substitution tables providing functionally similar amino acids are well known in the art. See, for example, Davis et al., “Basic Methods in Molecular Biology” Appleton & Lange, Norwalk, Conn. (1994). Such conservatively modified variants are in addition to and do not exclude polymorphic variants, interspecies homologs, and alleles of the disclosure.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

The isolated variant proteins can be purified from cells that naturally express it, purified from cells that have been altered to express it (recombinant), or synthesized using known protein synthesis methods. For example, a nucleic acid molecule encoding the variant polypeptide is cloned into an expression vector, the expression vector introduced into a host cell and the variant protein expressed in the host cell. The variant protein can then be isolated from the cells by an appropriate purification scheme using standard protein purification techniques. Many of these techniques are described in detail below.

The methods include amino acids that include an amino acid sequence that is at least about 70% identical to one that is set forth herein, more preferably at least 75%, still more preferably at least 80%, more preferably at least 85%, 85.5% 86%, 86.5% 87%, 87.5% 88%, 88.5%, 89%, 89.5% still more preferably at least 90%, 90.5%, 91%, 91.5% 92%, 92.5%, 93%, 94.5%, 94%, 94.5% and even more preferably at least about 95%, 95.5%, 96%, 96.5%, 97%, 97.5%, 98%, 98.5%, 99%, 95.5%, 100% identical (or any percentage in between) to an exemplified nucleotide sequence. The sequence may be modified as described previously, so long as the polypeptide is capable of inducing the generation of a protective response.

The variant proteins used in the present methods can be attached to heterologous sequences to form chimeric or fusion proteins. Such chimeric and fusion proteins comprise a variant protein fused in-frame to a heterologous protein having an amino acid sequence not substantially homologous to the variant protein. The heterologous protein can be fused to the N-terminus or C-terminus of the variant protein.

A chimeric or fusion protein can be produced by standard recombinant DNA techniques. For example, DNA fragments coding for the different protein sequences are ligated together in-frame in accordance with conventional techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see Ausubel et al., Current Protocols in Molecular Biology, 1992). Moreover, many expression vectors are commercially available that already encode a fusion moiety (e.g., a GST protein). A variant protein-encoding nucleic acid can be cloned into such an expression vector such that the fusion moiety is linked in-frame to the variant protein.

Polypeptides sometimes contain amino acids other than the 20 amino acids commonly referred to as the 20 naturally occurring amino acids. Further, many amino acids, including the terminal amino acids, may be modified by natural processes, such as processing and other post-translational modifications, or by chemical modification techniques well known in the art. Common modifications that occur naturally in polypeptides are described in basic texts, detailed monographs, and the research literature, and they are well known to those of skill in the art. Accordingly, the variant peptides of the present disclosure also encompass derivatives or analogs in which a substituted amino acid residue is not one encoded by the genetic code, in which a substituent group is included, in which the mature polypeptide is fused with another compound, such as a compound to increase the half-life of the polypeptide (for example, polyethylene glycol), or in which the additional amino acids are fused to the mature polypeptide, such as a leader or secretory sequence or a sequence for purification of the mature polypeptide or a pro-protein sequence.

Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

The present methods further provide functional fragments of the nucleic acid molecules and polypeptides including variant proteins of the polypeptide, in addition to proteins and peptides that comprise and consist of such fragments, provided that such fragments act as an antigen and/or provide treatment for and/or protection against Borrelia burgdorferi.

As used herein, the term “subunit” refers to a portion of the microorganism which provides protection and may itself be antigenic, i.e., capable of inducing an immune response in an animal. The term should be construed to include subunits which are obtained by both recombinant and biochemical methods.

In one embodiment, a method of identifying protective sequences of the virus or nucleic acids that elicit protection is provided. This method also includes fragments, derivatives, or homologs of the nucleic acid molecule. In one aspect, the method comprises administering to a test animal such sequences. The test and control animals are subsequently challenged with an infectious amount of a microorganism that causes the disease. Various methods and techniques for determining whether protection is provided are known to those skilled in the art, including but not limited to, observing a difference between the test and control animal in the symptoms of the disease, for example. A decrease in any of the symptoms observed in the test animal compared to the control animal indicates that protective molecule(s) provide a degree of protection against disease. Similar symptoms or an increase in any of the symptoms observed in the test animal compared to those observed in the control animal indicate that the protective molecule(s) do not provide protection.

In another aspect, determining whether the molecules provided protection against Borrelia burgdorferi includes determining the presence or absence of challenge disease in the test animal by electron microscopy or antibody or assays such as the fluorescent focusing neutralizing (FFN) test or Western blot assay may be used. PCR methods may be used to determine if the protective molecule is present. Northern blotting can detect the presence of diagnostic sequences. In another aspect, an ELISA or similar assay, such as a hemagglutinin inhibition assay are the types of many varied assays that can determine if the protective molecule is effective. The ELISA or enzyme linked immunoassay has been known since 1971. In general, antigens solubilized in a buffer are coated on a plastic surface. When serum is added, antibodies can attach to the antigen on the solid phase. The presence or absence of these antibodies can be demonstrated when conjugated to an enzyme. Adding the appropriate substrate will detect the amount of bound conjugate which can be quantified. A common ELISA assay is one which uses biotinylated anti-(protein) polyclonal antibodies and an alkaline phosphatase conjugate. For example, an ELISA used for quantitative determination of protein levels can be an antibody sandwich assay, which utilizes polyclonal rabbit antibodies obtained commercially. The antibody is conjugated to alkaline phosphatases for detection. In another example, an ELISA assay to detect trypsin or trypsinogen uses biotinylated anti-trypsin or anti-trypsinogen polyclonal antibodies and a streptavidin-alkaline phosphatase conjugate.

Clearly, many such methods are available to one skilled in the art to ascertain if the molecule provides protection and provides protection at the levels administered to the animal.

The nucleic acid molecule, polypeptide or fragment thereof, when administered to the subject animal produces a protective response to Borrelia burgdorferi. A protective response is elicited in the animal. The subject animal may or may not produce antibodies in response, but the animal will have decreased morbidity or mortality resulting from administration of the vaccine, and as described further herein. The terms “protecting”, “protection”, “protective immunity” or “protective immune response,” as used herein, are intended to mean that the host subject animal mounts an active immune response to the vaccine or polypeptides of the present disclosure, such that upon exposure to disease challenge, the subject animal is able to combat the infection. Thus, a protective immune response will decrease the incidence of morbidity and mortality from exposure to the microorganism among a host animal. The subject animal will be protected from subsequent exposure to the disease-causing agent. In an embodiment, the animal may be protected by treating the animal which has already been exposed to the disease-causing agent by administration of the vaccine or polypeptide after such exposure. In such an instance there is also shown to be a lessening of morbidity and mortality. Those skilled in the art will understand that in a commercial animal setting, the production of a protective immune response may be assessed by evaluating the effects of vaccination on the herd as a whole, e.g., there may still be morbidity and mortality in a minority of vaccinated animals. Furthermore, protection also includes a lessening in severity of any gross or histopathological changes and/or of symptoms of the disease, as compared to those changes or symptoms typically caused by the isolate in similar animals which are unprotected (i.e., relative to an appropriate control). Thus, a protective immune response will decrease the symptoms of the disease compared to a control animal.

A “construct” is a package of genetic material inserted into the genome of a cell via various techniques. A “vector” is any means for the transfer of a nucleic acid into a host cell. A vector may be a replicon to which another DNA segment may be attached so as to bring about the replication of the attached segment. A “replicon” is any genetic element (e.g., plasmid, phage, cosmid, chromosome, virus) that functions as an autonomous unit of DNA or RNA replication in vivo, i.e., capable of replication under its own control. The term “vector” includes both viral and nonviral means for introducing the nucleic acid into a cell in vitro, ex vivo or in vivo. Viral vectors include alphavirus, retrovirus, adeno-associated virus, pox, baculovirus, vaccinia, herpes simplex, Epstein-Barr, rabies virus, vesicular stomatitis virus, and adenovirus vectors. Non-viral vectors include, but are not limited to plasmids, liposomes, electrically charged lipids (cytofectins), DNA- or RNA protein complexes, and biopolymers. In addition to a nucleic acid, a vector may also contain one or more regulatory regions, and/or selectable markers useful in selecting, measuring, and monitoring nucleic acid transfer results (transfer to which tissues, duration of expression, etc.).

A “cassette” refers to a segment of DNA that can be inserted into a vector at specific restriction sites. The segment of DNA encodes a polypeptide of interest or produces RNA, and the cassette and restriction sites are designed to ensure insertion of the cassette in the proper reading frame for transcription and translation.

A nucleic acid molecule is introduced into a cell when it is inserted in the cell. A cell has been “transfected” by exogenous or heterologous DNA or RNA when such DNA or RNA has been introduced inside the cell.

Once the gene is engineered to contain desired features, such as the desired subcellular localization sequences, it may then be placed into an expression vector by standard methods. The selection of an appropriate expression vector will depend upon the method of introducing the expression vector into host cells. A typical expression vector contains prokaryotic DNA elements coding for a bacterial origin of replication and an antibiotic resistance gene to provide for the growth and selection of the expression vector in the bacterial host; a cloning site for insertion of an exogenous DNA sequence; eukaryotic DNA elements that control initiation of transcription of the exogenous gene; and DNA elements that control the processing of transcripts, such as transcription termination/polyadenylation sequences. It also can contain such sequences as are needed for the eventual integration of the vector into the host chromosome.

By “promoter” is meant a regulatory region of DNA capable of regulating the transcription of a sequence linked thereto. It usually comprises a TATA box capable of directing RNA polymerase II to initiate RNA synthesis at the appropriate transcription initiation site for a particular coding sequence. The promoter is the minimal sequence sufficient to direct transcription in a desired manner. The term “regulatory region” is also used to refer to the sequence capable of initiating transcription in a desired manner.

A nucleic acid molecule may be used in conjunction with its own or another promoter. In one embodiment, a selection marker and a nucleic acid molecule of interest can be functionally linked to the same promoter. In another embodiment, they can be functionally linked to different promoters. In yet third and fourth embodiments, the expression vector can contain two or more genes of interest that can be linked to the same promoter or different promoters. For example, one promoter can be used to drive a nucleic acid molecule of interest and the selectable marker, or a different promoter used for one or each. These other promoter elements can be those that are constitutive or sufficient to render promoter-dependent gene expression controllable as being cell-type specific, tissue-specific or time or developmental stage specific, or being inducible by external signals or agents. Such elements may be located in the 5′ or 3′ regions of the gene. Although the additional promoter may be the endogenous promoter of a structural gene of interest, the promoter can also be a foreign regulatory sequence. Promoter elements employed to control expression of product proteins and the selection gene can be any host-compatible promoters. These can be plant gene promoters, such as, for example, the ubiquitin promoter (European patent application no. 0 342 926); the promoter for the small subunit of ribulose-1,5-bis-phosphate carboxylase (ssRUBISCO) (Coruzzi et al., 1984; Broglie et al., 1984); or promoters from the tumor-inducing plasmids from Agrobacterium tumefaciens, such as the nopaline synthase, octopine synthase and mannopine synthase promoters (Velten and Schell, 1985) that have plant activity; or viral promoters such as the cauliflower mosaic virus (CaMV) 19S and 35S promoters (Guilley et al., 1982; Odell et al., 1985), the figwort mosaic virus FLt promoter (Maiti et al., 1997) or the coat protein promoter of TMV (Grdzelishvili et al., 2000). Alternatively, plant promoters such as heat shock promoters for example soybean hsp 17.5-E (Gurley et al., 1986); or ethanol-inducible promoters (Caddick et al., 1998) may be used. See International Patent Application No. WO 91/19806 for a review of illustrative plant promoters suitably employed.

A promoter can additionally comprise other recognition sequences generally positioned upstream or 5′ to the TATA box, referred to as upstream promoter elements, which influence the transcription initiation rate. It is recognized that having identified the nucleotide sequences for a promoter region, it is within the state of the art to isolate and identify further regulatory elements in the 5′ region upstream from the particular promoter region identified herein. Thus, the promoter region is generally further defined by comprising upstream regulatory elements such as those responsible for tissue and temporal expression of the coding sequence, enhancers and the like.

Tissue-preferred promoters can be utilized to target enhanced transcription and/or expression within a particular tissue. When referring to preferential expression, what is meant is expression at a higher level in the particular tissue than in other tissue. Examples of these types of promoters include seed preferred expression such as that provided by the phaseolin promoter (Bustos et al. (1989) The Plant Cell Vol. 1, 839-853). For dicots, seed-preferred promoters include, but are not limited to, bean β-phaseolin, napin, β-conglycinin, soybean lectin, cruciferin, and the like. For monocots, seed-preferred promoters include, but are not limited to, maize 15 kDa zein, 22 kDa zein, 27 kDa zein, γ-zein, waxy, shrunken 1, shrunken 2, an Ltp1 (See, for example, U.S. Pat. No. 7,550,579), an Ltp2 (Opsahl-Sorteberg, H-G. et al., (2004) Gene 341:49-58 and U.S. Pat. No. 5,525,716), and oleosin genes. See also WO 00/12733, where seed-preferred promoters from end1 and end2 genes are disclosed. Seed-preferred promoters also include those promoters that direct gene expression predominantly to specific tissues within the seed such as, for example, the endosperm-preferred promoter of γ-zein, the cryptic promoter from tobacco (Fobert et al. (1994) “T-DNA tagging of a seed coat-specific cryptic promoter in tobacco” Plant J. 4: 567-577), the P-gene promoter from corn (Chopra et al. (1996) “Alleles of the maize P gene with distinct tissue specificities encode Myb-homologous proteins with C-terminal replacements” Plant Cell 7:1149-1158, Erratum in Plant Cell 1997, 1:109), the globulin-1 promoter from corn (Belanger and Kriz (1991) “Molecular basis for Allelic Polymorphism of the maize Globulin-1 gene” Genetics 129: 863-972 and GenBank accession No. L22344), promoters that direct expression to the seed coat or hull of corn kernels, for example the pericarp-specific glutamine synthetase promoter (Muhitch et al., (2002) “Isolation of a Promoter Sequence From the Glutamine Synthetase₁₋₂ Gene Capable of Conferring Tissue-Specific Gene Expression in Transgenic Maize” Plant Science 163:865-872 and GenBank accession number AF359511) and to the embryo (germ) such as that disclosed at U.S. Pat. No. 7,169,967. When referring to a seed or an embryo preferred promoter is meant that it expresses an operably linked sequence to a higher degree in seed or embryo tissue than in other plant tissue. It may express during seed or embryo development, along with expression at other stages, may express strongly during seed or embryo development and to a much lesser degree at other times.

The range of available promoters includes inducible promoters. An inducible regulatory element is one that is capable of directly or indirectly activating transcription of one or more DNA sequences or genes in response to an inducer. In the absence of an inducer the DNA sequences or genes will not be transcribed. Typically, the protein factor that binds specifically to an inducible regulatory element to activate transcription is present in an inactive form which is then directly or indirectly converted to the active form by the inducer. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. A cell containing an inducible regulatory element may be exposed to an inducer by externally applying the inducer to the cell or plant such as by spraying, watering, heating or similar methods.

Any inducible promoter can be used. See Ward et al. Plant Mol. Biol. 22: 361-366 (1993). Exemplary inducible promoters include ecdysone receptor promoters, U.S. Pat. No. 6,504,082; promoters from the ACE1 system which responds to copper (Mett et al. PNAS 90: 4567-4571 (1993)); In2-1 and In2-2 gene from maize which respond to benzenesulfonamide herbicide safeners (U.S. Pat. No. 5,364,780; Hershey et al., Mol. Gen. Genetics 227: 229-237 (1991) and Gatz et al., Mol. Gen. Genetics 243: 32-38 (1994)); Tet repressor from Tn10 (Gatz et al., Mol. Gen. Genet. 227: 229-237 (1991); or from a steroid hormone gene, the transcriptional activity of which is induced by a glucocorticosteroid hormone. Schena et al., Proc. Natl. Acad. Sci. U.S.A. 88: 10421 (1991); the maize GST promoter, which is activated by hydrophobic electrophilic compounds that are used as pre-emergent herbicides; and the tobacco PR-la promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156).

Other components of the vector may be included, also depending upon intended use of the gene. Examples include selectable markers, targeting or regulatory sequences, stabilizing or leader sequences, introns, etc. General descriptions and examples of plant expression vectors and reporter genes can be found in Gruber, et al., “Vectors for Plant Transformation” in Method in Plant Molecular Biology and Biotechnology, Glick et al eds; CRC Press pp. 89-119 (1993). The selection of an appropriate expression vector will depend upon the host and the method of introducing the expression vector into the host. The expression cassette will also include at the 3′ terminus of the heterologous nucleotide sequence of interest, a transcriptional and translational termination region functional in plants.

The expression vector can optionally also contain a signal sequence located between the promoter and the gene of interest and/or after the gene of interest. A signal sequence is a nucleotide sequence, translated to give an amino acid sequence, which is used by a cell to direct the protein or polypeptide of interest to be placed in a particular place within or outside the eukaryotic cell. Many signal sequences are known in the art. See, for example Becker et al., (1992) Plant Mol. Biol. 20:49, Knox, C., et al., “Structure and Organization of Two Divergent Alpha-Amylase Genes from Barley”, Plant Mol. Biol. 9:3-17 (1987), Lerner et al., (1989) Plant Physiol. 91:124-129, Fontes et al., (1991) Plant Cell 3:483-496, Matsuoka et al., (1991) Proc. Natl. Acad. Sci. 88:834, Gould et al., (1989) J. Cell. Biol. 108:1657, Creissen et al., (1991) Plant J. 2:129, Kalderon, et al., (1984) “A short amino acid sequence able to specify nuclear location,” Cell 39:499-509, Steifel, et al., (1990) “Expression of a maize cell wall hydroxyproline-rich glycoprotein gene in early leaf and root vascular differentiation” Plant Cell 2:785-793. When targeting the protein to the cell wall and/or apoplast use of a signal sequence is necessary. One example is the barley alpha-amylase signal sequence. Rogers, J. C. (1985) “Two barley alpha-amylase gene families are regulated differently in aleurone cells” J. Biol. Chem. 260: 3731-3738.

In those instances where it is desirable to have the expressed product of the heterologous nucleotide sequence directed to a particular organelle, particularly the plastid, amyloplast, or to the endoplasmic reticulum, or secreted at the cell's surface or extracellularly, the expression cassette can further comprise a coding sequence for a transit peptide. Such transit peptides are well known in the art and include, but are not limited to, the transit peptide for the acyl carrier protein, the small subunit of RUBISCO, plant EPSP synthase, Zea mays Brittle-1 chloroplast transit peptide (Nelson et al. Plant Physiol 117(4):1235-1252 (1998); Sullivan et al. Plant Cell 3(12):1337-48; Sullivan et al., Planta (1995) 196(3):477-84; Sullivan et al., J. Biol. Chem. (1992) 267(26):18999-9004) and the like. One skilled in the art will readily appreciate the many options available in expressing a product to a particular organelle. Use of transit peptides is well known (e.g., see U.S. Pat. Nos. 5,717,084; 5,728,925). A protein may be targeted to the endoplasmic reticulum of the plant cell. This may be accomplished by use of a localization sequence, such as KDEL. This sequence (Lys-Asp-Glu-Leu) contains the binding site for a receptor in the endoplasmic reticulum. (Munro et al., (1987) “A C-terminal signal prevents secretion of luminal ER proteins.” Cell. 48:899-907. There are a wide variety of endoplasmic reticulum retention signal sequences available to one skilled in the art and the KDEL sequence is one example. Another example is HDEL (His-Asp-Glu-Leu). See, for example, Kumar et al. which discuses methods of producing a variety of endoplasmic reticulum proteins. Kumar et al. (2017) “prediction of endoplasmic reticulum resident proteins using fragmented amino acid composition and support vector machine” Peer J. doi: 10.7717/peerj.3561.

Retaining the protein in the vacuole is another example. Signal sequences to accomplish this are well known. For example, Raikhel U.S. Pat. No. 5,360,726 shows a vacuole signal sequence as does Warren et al at U.S. Pat. No. 5,889,174. Vacuolar targeting signals may be present either at the amino-terminal portion, (Holwerda et al., (1992) The Plant Cell, 4:307-318, Nakamura et al., (1993) Plant Physiol., 101:1-5), carboxy-terminal portion, or in the internal sequence of the targeted protein. (Tague et al., (1992) The Plant Cell, 4:307-318, Saalbach et al. (1991) The Plant Cell, 3:695-708). Additionally, amino-terminal sequences in conjunction with carboxy-terminal sequences are responsible for vacuolar targeting of gene products (Shinshi et al. (1990) Plant Molec. Biol. 14:357-368).

The termination region can be native with the DNA sequence of interest or can be derived from another source. Convenient termination regions are available from the Ti-plasmid of A, tumefaciens, such as the octopine synthase (MacDonald et al., (1991) Nuc. Acids Res. 19(20)5575-5581) and nopaline synthase termination regions (Depicker et al., (1982) Mol. and Appl. Genet. 1:561-573 and Shaw et al. (1984) Nucleic Acids Research Vol. 12, No. 20 pp7831-7846 (nos). Examples of various other terminators include the pin II terminator from the protease inhibitor II gene from potato (An, et al. (1989) Plant Cell 1, 115-122. See also, Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

Many variations on the promoters, selectable markers, signal sequences, leader sequences, termination sequences, introns, enhancers and other components of the vector are available to one skilled in the art.

Plant is used broadly herein to include any plant at any stage of development, or to part of a plant, including a plant cutting, a plant cell culture, a plant organ, a plant seed, and a plantlet. In other words, the term plant refers to the entire plant or plant material or plant part or plant tissue or plant cell including a collection of plant cells. Plant seed parts, for example, include the pericarp or kernel, the embryo or germ, and the endoplasm. A plant cell is the structural and physiological unit of the plant, comprising a protoplast and a cell wall. A plant cell can be in the form of an isolated single cell or aggregate of cells such as a friable callus, or a cultured cell, or can be part of a higher organized unit, for example, a plant tissue, plant organ, or plant. Thus, a plant cell can be a protoplast, a gamete producing cell, or a cell or collection of cells that can regenerate into a whole plant. A plant tissue or plant organ can be a seed, protoplast, callus, or any other groups of plant cells that is organized into a structural or functional unit. Particularly useful parts of a plant include harvestable parts and parts useful for propagation of progeny plants. A harvestable part of a plant can be any useful part of a plant, for example, flowers, pollen, seedlings, tubers, leaves, stems, fruit, seeds, roots, and the like. A part of a plant useful for propagation includes, for example, seeds, fruits, cuttings, seedlings, tubers, rootstocks, and the like. In an embodiment, the tissue culture will preferably be capable of regenerating plants. Preferably, the regenerable cells in such tissue cultures will be embryos, protoplasts, meristematic cells, callus, pollen, leaves, anthers, roots, root tips, silk, flowers, kernels, ears, cobs, husks or stalks. Still further, plants may be regenerated from the tissue cultures.

Any plant species may be used, whether monocotyledonous or dicotyledonous, including but not limited to corn (Zea mays), canola (Brassica napus, Brassica rapa ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum), soybean (Glycine max), tobacco (Nicotiana tabacum), potato (Solanum tuberosum), peanuts (Arachis hypogaea), cotton (Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), oats (Avena), barley (Hordeum), vegetables, ornamentals, and conifers. Vegetables include tomatoes (Lycopersicon esculentum), lettuce (e.g., Lactuca sativa), green beans (Phaseolus vulgaris), lima beans (Phaseolus limensis), peas (Lathyrus spp.) and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers which may be employed in practicing the present disclosure include, for example, pines such as loblolly pine (Pinus taeda), slash pine (Pinus elliotii), ponderosa pine (Pinus ponderosa), lodgepole pine (Pinus contotta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis) algae, or Lemnoideae (aka Duckweed). An embodiment provides the plant is maize.

Various methods of transformation or transfection are currently available. As newer methods are available to transform crops or other host cells they may be directly applied. Accordingly, a wide variety of methods have been developed to insert a DNA sequence into the genome of a host cell to obtain the transcription or transcript and translation of the sequence to effect phenotypic changes in the organism. Thus, any method which provides for efficient transformation/transfection may be employed. Notably, the transformation vector comprising the sequence operably linked to a heterologous nucleotide sequence in an expression cassette can also contain at least one additional nucleotide sequence for a gene to be cotransformed into the organism. Alternatively, the additional sequence(s) can be provided on another transformation vector.

Methods for introducing expression vectors into plant tissue available to one skilled in the art are varied and will depend on the plant selected. Procedures for transforming a wide variety of plant species are well known and described throughout the literature. (See, for example, Miki and McHugh (2004) Biotechnol. 107, 193-232; Klein et al. (1992) Biotechnology (N Y) 10, 286-291; and Weising et al. (1988) Annu. Rev. Genet. 22, 421-477). For example, the DNA construct may be introduced into the genomic DNA of the plant cell using techniques such as microprojectile-mediated delivery (Klein et al. 1992, supra), electroporation (Fromm et al., 1985 Proc. Natl. Acad. Sci. USA 82, 5824-5828), polyethylene glycol (PEG) precipitation (Mathur and Koncz, 1998 Methods Mol. Biol. 82, 267-276), direct gene transfer (WO 85/01856 and EP-A-275 069), in vitro protoplast transformation (U.S. Pat. No. 4,684,611), and microinjection of plant cell protoplasts or embryogenic callus (Crossway, A. (1985) Mol. Gen. Genet. 202, 179-185). Agrobacterium transformation methods of Ishida et al. (1996) and also described in U.S. Pat. No. 5,591,616 are yet another option. Co-cultivation of plant tissue with Agrobacterium tumefaciens is a variation, where the DNA constructs are placed into a binary vector system (Ishida et al., 1996 Nat. Biotechnol. 14, 745-750). The virulence functions of the Agrobacterium tumefaciens host will direct the insertion of the construct into the plant cell DNA when the cell is infected by the bacteria. See, for example, Fraley et al. (1983) Proc. Natl. Acad. Sci. USA, 80, 4803-4807. Agrobacterium is primarily used in dicots, but monocots including maize can be transformed by Agrobacterium. See, for example, U.S. Pat. No. 5,550,318. In one of many variations on the method, Agrobacterium infection of corn can be used with heat shocking of immature embryos (Wilson et al. U.S. Pat. No. 6,420,630) or with antibiotic selection of Type II callus (Wilson et al., U.S. Pat. No. 6,919,494).

Rice transformation is described by Hiei et al. (1994) Plant J. 6, 271-282 and Lee et al. (1991) Proc. Nat. Acad. Sci. USA 88, 6389-6393. Standard methods for transformation of canola are described by Moloney et al. (1989) Plant Cell Reports 8, 238-242. Corn transformation is described by Fromm et al. (1990) Biotechnology (N Y) 8, 833-839 and Gordon-Kamm et al. (1990) supra. Wheat can be transformed by techniques similar to those used for transforming corn or rice. Sorghum transformation is described by Casas et al. (Casas et al. (1993). Transgenic sorghum plants via microprojectile bombardment. Proc. Natl. Acad. Sci. USA 90, 11212-11216) and barley transformation is described by Wan and Lemaux (Wan and Lemaux (1994) Generation of large numbers of independently transformed fertile barley plants. Plant Physiol. 104, 37-48). Soybean transformation is described in a number of publications, including U.S. Pat. No. 5,015,580.

In one method, the Agrobacterium transformation methods of Ishida et al. (1996) and also described in U.S. Pat. No. 5,591,616, are generally followed, with modifications that the inventors have found improve the number of transformants obtained. The Ishida method uses the A188 variety of maize that produces Type I callus in culture. In an embodiment the Hill maize line is used which initiates Type II embryogenic callus in culture (Armstrong et al., 1991).

While Ishida recommends selection on phosphinothricin when using the bar or pat gene for selection, another preferred embodiment provides use of bialaphos instead. In general, as set forth in the 5,591,616 patent, and as outlined in more detail below, dedifferentiation is obtained by culturing an explant of the plant on a dedifferentiation-inducing medium for not less than seven days, and the tissue during or after dedifferentiation is contacted with Agrobacterium having the gene of interest. The cultured tissue can be callus, an adventitious embryo-like tissue or suspension cells, for example. In this preferred embodiment, the suspension of Agrobacterium has a cell population of 10⁶ to 10¹¹ cells/ml and are contacted for three to ten minutes with the tissue, or continuously cultured with Agrobacterium for not less than seven days. The Agrobacterium can contain plasmid pTOK162, with the gene of interest between border sequences of the T region of the plasmid, or the gene of interest may be present in another plasmid-containing Agrobacterium. The virulence region may originate from the virulence region of a Ti plasmid or Ri plasmid. The bacterial strain used in the Ishida protocol is LBA4404 with the 40 kb super binary plasmid containing three vir loci from the hypervirulent A281 strain. The plasmid has resistance to tetracycline. The cloning vector cointegrates with the super binary plasmid. Since the cloning vector has an E. coli specific replication origin, but not an Agrobacterium replication origin, it cannot survive in Agrobacterium without cointegrating with the super binary plasmid. Since the LBA4404 strain is not highly virulent, and has limited application without the super binary plasmid, the inventors have found in yet another embodiment that the EHA101 strain is preferred. It is a disarmed helper strain derived from the hypervirulent A281 strain. The cointegrated super binary/cloning vector from the LBA4404 parent is isolated and electroporated into EHA101, selecting for spectinomycin resistance. The plasmid is isolated to assure that the EHA101 contains the plasmid. EHA101 contains a disarmed pTi that carries resistance to kanamycin. See, Hood et al. (1986).

Further, the Ishida protocol as described provides for growing fresh culture of the Agrobacterium on plates, scraping the bacteria from the plates, and resuspending in the co-culture medium as stated in the 5,591,616 patent for incubation with the maize embryos. This medium includes 4.3 g MS salts, 0.5 mg nicotinic acid, 0.5 mg pyridoxine hydrochloride, 1.0 ml thiamine hydrochloride, casamino acids, 1.5 mg 2,4-D, 68.5 g sucrose and 36 g glucose per liter, all at a pH of 5.8. In a further preferred method, the bacteria are grown overnight in a 1 ml culture and then a fresh 10 ml culture is re-inoculated the next day when transformation is to occur. The bacteria grow into log phase and are harvested at a density of no more than OD₆₀₀=0.5, preferably between 0.2 and 0.5. The bacteria are then centrifuged to remove the media and resuspended in the co-culture medium. Since Hill is used, medium preferred for Hill is used. This medium is described in considerable detail by Armstrong and Green (1985). The resuspension medium is the same as that described above. All further Hill media are as described in Armstrong and Green (1985). The result is redifferentiation of the plant cells and regeneration into a plant. Redifferentiation is sometimes referred to as dedifferentiation, but the former term more accurately describes the process where the cell begins with a form and identity, is placed on a medium in which it loses that identity and becomes “reprogrammed” to have a new identity. Thus, the scutellum cells become embryogenic callus.

A transgenic plant may be produced that contains an introduced nucleic acid molecule encoding the polypeptide.

When referring to introduction of a nucleotide sequence into a plant is meant to include transformation into the cell, as well as crossing a plant having the sequence with another plant, so that the second plant contains the heterologous sequence, as in conventional plant breeding techniques. Such breeding techniques are well known to one skilled in the art. This can be accomplished by any means known in the art for breeding plants such as, for example, cross pollination of the transgenic plants that are described above with other plants, and selection for plants from subsequent generations which express the amino acid sequence. The plant breeding methods used herein are well known to one skilled in the art. For a discussion of plant breeding techniques, see Poehlman (1995) Breeding Field Crops. AVI Publication Co., Westport Conn, 4^{th} Edit.). Many crop plants useful in this method are bred through techniques that take advantage of the plant's method of pollination. A plant is self-pollinating if pollen from one flower is transferred to the same or another flower of the same plant. A plant is cross-pollinating if the pollen comes from a flower on a different plant. For example, in Brassica, the plant is normally self-sterile and can only be cross-pollinated unless, through discovery of a mutant or through genetic intervention, self-compatibility is obtained. In self-pollinating species, such as rice, oats, wheat, barley, peas, beans, soybeans, tobacco and cotton, the male and female plants are anatomically juxtaposed. During natural pollination, the male reproductive organs of a given flower pollinate the female reproductive organs of the same flower. Maize plants (Zea mays L.) can be bred by both self-pollination and cross-pollination techniques. Maize has male flowers, located on the tassel, and female flowers, located on the ear, on the same plant. It can self or cross-pollinate.

Pollination can be by any means, including but not limited to hand, wind or insect pollination, or mechanical contact between the male fertile and male sterile plant. For production of hybrid seeds on a commercial scale in most plant species pollination by wind or by insects is preferred. Stricter control of the pollination process can be achieved by using a variety of methods to make one plant pool male sterile, and the other the male fertile pollen donor. This can be accomplished by hand detasseling, cytoplasmic male sterility, or control of male sterility through a variety of methods well known to the skilled breeder. Examples of more sophisticated male sterility systems include those described by Brar et al., U.S. Pat. Nos. 4,654,465 and 4,727,219 and Albertsen et al., U.S. Pat. Nos. 5,859,341 and 6,013,859.

Backcrossing methods may be used to introduce the gene into the plants. This technique has been used for decades to introduce traits into a plant. An example of a description of this and other plant breeding methodologies that are well known can be found in references such as Neal (1988). In a typical backcross protocol, the original variety of interest (recurrent parent) is crossed to a second variety (nonrecurrent parent) that carries the single gene of interest to be transferred. The resulting progeny from this cross are then crossed again to the recurrent parent and the process is repeated until a plant is obtained wherein essentially all of the desired morphological and physiological characteristics of the recurrent parent are recovered in the converted plant, in addition to the single transferred gene from the nonrecurrent parent.

Selection and propagation techniques described above can yield a plurality of transgenic plants that are harvested in a conventional manner. The plant or any parts expressing the recombinant polypeptide can be used in a commercial process, or the polypeptide extracted. When using the plant or part itself, it can, for example, be made into flour and then applied in the commercial process. Polypeptide extraction from biomass can be accomplished by known methods. Downstream processing for any production system refers to all unit operations after product synthesis, in this case protein production in transgenic seed (Kusnadi, A. R., Nikolov, Z. L., Howard, J. A., 1997. Biotechnology and Bioengineering. 56:473-484). For example, seed can be processed either as whole seed ground into flour or, fractionated and the germ separated from the hulls and endosperm. If germ is used, it is usually defatted using an extraction process and the remaining crushed germ ground into a meal or flour. In some cases, the germ is used directly in the process or the protein can be extracted (See, e.g., WO 98/39461). Extraction is generally made into aqueous buffers at specific pH to enhance recombinant protein extraction and minimize native seed protein extraction. Subsequent protein concentration or purification can follow.

The compositions and process described here are also for producing and administering a vaccine that protects an animal from Borrelia burgdorferi.

As used herein, the term “vaccine” refers to a pharmaceutical composition comprising at least one protective molecule, that induces protective response in a subject and possibly, but not necessarily, one or more additional components that enhance the activity of the active component. A vaccine may additionally comprise further components typical to pharmaceutical compositions. In another form, the immunologically active component of a vaccine may comprise appropriate elements of the organisms (subunit vaccines) whereby these elements are generated either by destroying the whole organism or the growth cultures of such microorganisms and subsequent purification steps yielding in the desired structure(s), or by synthetic processes induced by an appropriate manipulation of a suitable system such as, but not restricted to, bacteria, insects, mammalian, or other species, plus subsequent isolation and purification procedures or by induction of the synthetic processes in the animal needing a vaccine by direct incorporation of genetic material using suitable pharmaceutical compositions (polynucleotide vaccination). A vaccine may comprise one or simultaneously more than one of the elements described above.

The present vaccines may include a pharmaceutically acceptable carrier, excipient, carrier, stabilizer and/or diluent. Without intending to be limiting, examples include wetting agents and lubricating agents, preservative agents, lipids, stabilizers, solubilizers and emulsifiers. The carrier can be a solvent or dispersion medium containing, for example, water, ethanol, polyol (for example, glycerol, propylene glycol, liquid polyethylene glycol, and the like), suitable mixtures thereof and vegetable oils. One possible carrier is a physiological salt solution. Examples of stabilizers include, for example, glycerol/EDTA, carbohydrates (such as sorbitol, mannitol, trehalose, starch, sucrose, dextran or glucose), proteins (such as albumin or casein) and protein degradation products (e.g., partially hydrolyzed gelatin).

It is possible to provide an adjuvant in the vaccine. Adjuvants enhance the immunogenicity of an antigen but are not necessarily immunogenic themselves. Adjuvants may act by retaining the antigen locally near the site of administration to produce a depot effect facilitating a slow, sustained release of antigen to cells of the immune system. Adjuvants can also attract cells of the immune system to an antigen depot and stimulate such cells to elicit immune responses. Immunostimulatory agents or adjuvants have been used for many years to improve the host immune responses to, for example, vaccines. The vaccines of the present disclosure may be used in conjunction with adjuvants, for example, lipopolysaccharides, aluminum hydroxide and aluminum phosphate (alum), saponins complexed to membrane protein antigens (immune stimulating complexes), pluronic polymers with mineral oil, killed mycobacteria in mineral oil, Freund's complete adjuvant, bacterial products, such as muramyl dipeptide (MDP) and lipopolysaccharide (LPS), as well as lipid A, and liposomes. Desirable characteristics of ideal adjuvants may include: (1) lack of toxicity; (2) ability to stimulate a long-lasting immune response; (3) simplicity of manufacture and stability in long-term storage; (4) ability to elicit both CMI and HIR to antigens administered by various routes; (5) synergy with other adjuvants; (6) capability of selectively interacting with populations of antigen presenting cells (APC); (7) ability to specifically elicit appropriate T-cell helper 1 (TH 1) or TH 2 cell-specific immune responses; and (8) ability to selectively increase appropriate antibody isotype levels (for example, IgA) against antigens. An adjuvant used with the present compositions and methods need not possess all these characteristics to be used.

As used herein, “immunogenically effective amount” refers to an amount, which is effective in reducing, eliminating, treating, preventing or controlling the symptoms of the infections, diseases, disorders, or condition.

The quantity to be administered depends on the subject to be treated, including, for example, the capacity of the immune system of the individual to mount a protective response. Suitable regimes for initial administration and booster doses are also variable but may include an initial administration followed by subsequent administrations. For example, it may be desirable to provide for an initial administration of the vaccine followed by additional doses. The need to provide an effective amount of the protective molecule will also need to be balanced with cost of providing higher amounts of the protective molecule. A cost-effective vaccine is one in which the cost of producing it is less than the value one can obtain from using it. Measurement and determination of efficacy of any of the compositions and vaccines of the disclosure may be accomplished by any of the many methods available to one skilled in the art.

In one embodiment, a straightforward and quick method can be to perform a Western blot analysis of a sample candidate vaccine composition to quantitate the amount of polypeptide or fragment thereof that is present in the sample. In one embodiment, one compares the amount of polypeptide to a standard known to be effective with like polypeptides from other biotypes, and either prepares a vaccine where the level of polypeptide produced is at least at this standard or higher or may test the vaccine with a test animal.

The compounds described herein can be administered to a subject at therapeutically effective doses to prevent Lyme disease. The dosage will depend upon the subject receiving the vaccine as well as factors such as the size, weight, and age of the subject.

The precise amount of immunogenic composition of the disclosure to be employed in a formulation will depend on the route of administration and the nature of the subject (e.g., age, size, stage/level of disease), and should be decided according to the judgment of the practitioner and each subject's circumstances according to standard clinical techniques. An effective immunizing amount is that amount sufficient to treat or prevent Lyme disease in a subject.

Immunogenicity of a composition can be determined by monitoring the immune response of test subjects following immunization with the composition by use of any immunoassay known in the art. Generation of a humoral (antibody) response and/or cell-mediated immunity may be taken as an indication of an immune response.

The immune response of the test subjects can be analyzed by various approaches such as: the reactivity of the resultant immune serum to the immunogenic conjugate, as assayed by known techniques, e.g., enzyme linked immunosorbent assay (ELISA), immunoblots, immunoprecipitations, virus neutralization, etc.; or, by protection of immunized hosts from infection by the pathogen and/or attenuation of symptoms due to infection by the pathogen in immunized hosts as determined by any method known in the art, for assaying the levels of an infectious disease agent, e.g., the viral levels (for example, by culturing of a sample from the subject), or other technique known in the art. The levels of the infectious disease agent may also be determined by measuring the levels of the antigen against which the immunoglobulin was directed. A decrease in the levels of the infectious disease agent or an amelioration of the symptoms of the infectious disease indicates that the composition is effective.

The therapeutics of the invention can be tested in vitro for the desired therapeutic or prophylactic activity, prior to in vivo use. For example, in vitro assays that can be used to determine whether administration of a specific therapeutic is indicated include in vitro cell culture assays in which appropriate cells from a cell line or cells cultured from a subject having a particular disease or disorder are exposed to or otherwise administered a therapeutic, and the effect of the therapeutic on the cells is observed.

In addition, the therapeutics may be assayed by contacting the therapeutic to cells (either cultured from a subject or from a cultured cell line) that are susceptible to infection by the infectious disease agent but that are not infected with the infectious disease agent, exposing the cells to the infectious disease agent, and then determining whether the infection rate of cells contacted with the therapeutic was lower than the infection rate of cells not contacted with the therapeutic. Infection of cells with an infectious disease agent may be assayed by any method known in the art.

The therapeutics can also be assessed by measuring the level of the molecule against which the antibody is directed in the animal model and/or human subject at suitable time intervals before, during, or after therapy. Any change or absence of change in the amount of the molecule can be identified and correlated with the effect of the treatment on the subject. The level of the molecule can be determined by any method known in the art.

Following vaccination of an animal to Borrelia burgdorferi using the methods and compositions of the present invention, any binding assay known in the art can be used to assess the binding between the resulting antibody and the particular molecule. These assays may also be performed to select antibodies that exhibit a higher affinity or specificity for the particular antigen. As one measure of vaccine potency, an ELISA can be performed on a sample collected from an individual vaccinated to determine whether antibodies to a vaccine comprising the sequence, a derivative, a homologue or a variant or fragment thereof generated anti-polypeptide antibodies. The individual's sample is measured against a reference anti-polypeptide antibody. Analysis of symptoms and measurement of animal weight gain also demonstrated lessening of impact of the disease in the presence of a particular dose. Fluorescent focused neutralization assay is still another assay to detect serum neutralizing antibodies and analyze effectiveness of a vaccine and a particular dose.

When testing animals administered the vaccine, for example, measuring antibody response is also effective in determining efficacy of the vaccine. Sera may be collected and titer measured as the reciprocal of the maximal dilution at which hemagglutination is inhibited, as described in an example below. Other measurements post-administration of the vaccine can also be employed to determine effectiveness, whether pathological evaluation, isolation of the pathogen, measurement of symptoms, and overall health and weight gain of the subject.

Vaccine effectiveness may also be evaluated quantitatively (for example, a decrease in the percentage of diseased tissue as compared to an appropriate control group) or qualitatively (e.g., isolation of virus from blood, detection of virus antigen in a tissue sample by an assay method, etc.). The symptoms of the disease may be evaluated quantitatively (e.g., temperature/fever), semi-quantitatively (e.g., severity of distress), or qualitatively (e.g., the presence or absence of one or more symptoms or a reduction in severity of one or more symptoms). Clearly one skilled in the art has many different options available for measuring effectiveness of the vaccine. Protection periods of more than seven days after at least one challenge or exposure to the pathogenic microorganism have been achieved, and protection of at least two weeks, at least 20 days, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 days or more, have been achieved using the invention. Such protection periods are also provided when using the invention with other animals. The protective response is also shown here in an embodiment to be specific to the disease as opposed to another disease, and thus demonstrates specific memory.

Standard methods can be used to administer the vaccine including intranasal, oral and/or parenteral (e.g., intramuscular) administration. For example, the Borrelia burgdorferi OspA protein-containing vaccine can be administered intramuscularly one or more times. In another embodiment of the method, for example, the vaccine is administered orally one or more times. In an alternative embodiment oral administration can be followed by and/or precede administration of the vaccine at least once, intramuscularly. The maize grain can be made into a food product and fed to the animal, thereby reducing cost and loss of antigen that can occur through further processing.

The following numbered embodiments also form part of the present disclosure:

1. A method of producing a protective response to Borrelia burgdorferi in an animal, the method comprising: orally administering to the animal a composition comprising a plant or plant product comprising the OspA polypeptide of Borrelia burgdorferi, or a sequence having at least 90% sequence identity thereto or a functional fragment thereof, wherein a=a protective response to Borrelia burgdorferi is produced in the animal.

2. The method of embodiment 1, wherein the composition comprises seed or embryo of seed.

3. The method of embodiment 1 or embodiment 2, wherein the OspA polypeptide is expressed at levels of at least 10 mg/kg in seed of the plant.

4. The method of any one of embodiments 1-3, wherein the protective response comprises a serum antibody response in the animal.

5. The method of any one of embodiments 1-4, wherein the serum antibody response is at least 4 times greater than serum antibody response in an animal not administered the composition.

6. The method of any one of embodiments 1-5, wherein the OspA polypeptide encoding sequence is optimized for maize expression.

7. The method of any one of embodiments 1-6, wherein the OspA polypeptide is targeted to the apoplast or to the endoplasmic reticulum.

8. A vaccine comprising a plant-produced OspA polypeptide of Borrelia burgdorferi, the vaccine comprising a plant or plant product comprising a construct comprising: (a) a promoter preferentially directing expression to seed of a plant; (b) a nucleic acid molecule encoding the OspA polypeptide of Borrelia burgdorferi, or a sequence having at least 90% sequence identity thereto or a functional fragment operably linked to the promoter; and (c) a nucleic acid molecule targeting expression of the polypeptide to the apoplast or endoplasmic reticulum of the plant.

9 The vaccine of embodiment 8, wherein the plant product comprises seed or embryo of seed.

10. The vaccine of embodiment 8 or embodiment 9, wherein the OspA polypeptide is expressed at levels of at least 10 mg/kg in seed of the plant.

11. The vaccine of any one of embodiments 8-10, wherein the OspA polypeptide encoding sequence is optimized for maize expression.

12. The vaccine of any one of embodiments 8-11, wherein the construct comprises SEQ ID NO: 1, 3, 5, 7, or 9, or a sequence with at least 90% sequence identity thereto.

13. The vaccine of any one of embodiments 8-12, wherein the construct encodes SEQ ID NO: 2, 4, 6, 8, 10, or a sequence with at least 90% sequence identity thereto.

14. The vaccine of any one of embodiments 8-13, wherein the construct has a promotor set forth in SEQ ID NO: 11, or a sequence with at least 90% sequence identity thereto.

15. The vaccine of any one of embodiments 8-14, wherein the construct has a terminator set forth in SEQ ID NO: 12, or a sequence with at least 90% sequence identity thereto.

16. A method of expressing the OspA polypeptide of Borrelia burgdorferi or a functional fragment thereof, the method comprising introducing into a plant a construct comprising: (a) a promoter preferentially directing expression to seed of a plant; (b) a nucleic acid molecule encoding the OspA polypeptide of Borrelia burgdorferi, or a sequence having at least 90% sequence identity thereto or a functional fragment operably linked to the promoter; and (c) a nucleic acid molecule targeting expression of the polypeptide to the apoplast or endoplasmic reticulum of the plant.

17. The method of embodiment 16, wherein the OspA polypeptide is expressed at levels of at least 10 mg/kg in seed of the plant.

18. The method of embodiment 16 or embodiment 17, wherein the OspA polypeptide encoding sequence is optimized for maize expression.

19. The method of any one of embodiments 16-18, wherein the construct comprises SEQ ID NO: 1, 3, 5, 7, or 9, or a sequence with at least 90% sequence identity thereto.

20. The method of any one of embodiments 16-19, wherein the construct encodes SEQ ID NO: 2, 4, 6, 8, or 10, or a sequence with at least 90% sequence identity thereto.

21. The method of any one of embodiment 16-20, wherein the promotor is set forth in SEQ ID NO: 11, or a sequence with at least 90% sequence identity thereto.

22. The method of any one of embodiment 16-21, wherein the terminator is set forth in SEQ ID NO: 12, or a sequence with at least 90% sequence identity thereto.

It is to be understood that all terminology used herein is for the purpose of describing particular embodiments only and is not intended to be limiting in any manner or scope. For example, as used in this specification and the appended claims, the singular forms “a,” “an” and “the” can include plural referents unless the content clearly indicates otherwise. Similarly, the word “or” is intended to include “and” unless the context clearly indicate otherwise. The word “of” means any one member of a particular list and also includes any combination of members of that list. Further, all units, prefixes, and symbols may be denoted in its SI accepted form.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, it will be obvious that certain changes and modifications may be practiced within the scope of the appended embodiments.

The following examples are offered by way of illustration and not by way of limitation.

### EXAMPLES

### Example 1

### Construct Preparation

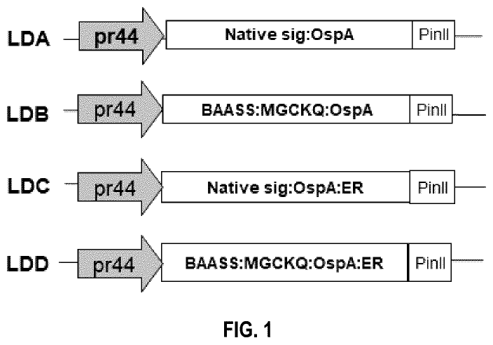

The sequence of the outer surface protein OspA from Borrelia burgdorferi (Genbank #NC_001857.2) was optimized for maize codon usage. The nucleotide sequence of the coding region was outsourced for commercial gene synthesis by GenScript. Four different constructs were prepared (FIG. 1) with varying subcellular targets.

Each antigen is targeted either to the apoplast/cell wall using the native signal sequence or a well-characterized barley alpha amylase sequence at the amino terminus or to the endoplasmic reticulum (ER) by the further addition of the KDEL signal sequence to the carboxy terminus. The native signal sequence was maintained in constructs LDA and LDC. The native signal sequence was replaced with a barley alpha amylase signal sequence (BAASS) in constructs LDB and LDD. In constructs LDC and LDD the amino acid sequence KDEL was added to the carboxy terminus for targeting to the endoplasmic reticulum (ER).

Constructs LDA and LDC incorporate the native OspA signal sequence to retain the lipidation signal sequence. In constructs LDB and LDD the junction between the BAASS plant apoplast/cell wall signal sequence was modified to create a plant lipidation sequence (MGCKQ). In all constructs the OspA coding region was synthesized for transfer into the maize transformation vector pSB11 using an NcoI site overlapping the initiating ATG and a PacI restriction site in the terminator region to add the coding region downstream of the promoter. All four constructs are under control of the synthetic pr44 promoter (SEQ ID NO: 11) targeting expression to the embryo. This promoter contains duplications of part of the well-characterized maize globulin-1 promoter that should increase expression levels. Each transcription unit also incorporated the terminator from potato proteinase inhibitor II.

### Plant Transformation

Maize transformation was carried out as previously described (Nat Biotechnol. 1996 Jun.;14(6):745-50) with modifications. In brief, the constructs were transferred into the LBA4404 Agrobacterium strain containing the vector pSB1 by a triparental mating procedure. The cointegrate DNA was then electroporated into Agrobacterium tumefaciens strain EHA101. Hill maize embryos roughly 1.5 to 3 mm in length were mixed with A. tumefaciens EHA101 with the appropriate vector for transformation. Plants from events selected on bialaphos were grown to maturity in the greenhouse and pollinated with Hill to produce T1 generation seed.

### Western Blot Analysis

To analyze expression in the T1 generation seed, 10 seed pools were ground and extracted in 1XPBS+0.05% tween then tested by western blot. The blot was incubated in anti-OspA mouse monoclonal antibodies LA2.2 or 184 overnight at a dilution of 1:1000 and developed with anti-mouse-alkaline phosphatase conjugate at a dilution of 1:2000 and BCIP-NBT liquid substrate. The positive control was 1.5 μg of purified E. coli OspA standard from Dr. Gomes-Soleki and the concentration was estimated visually using this standard.

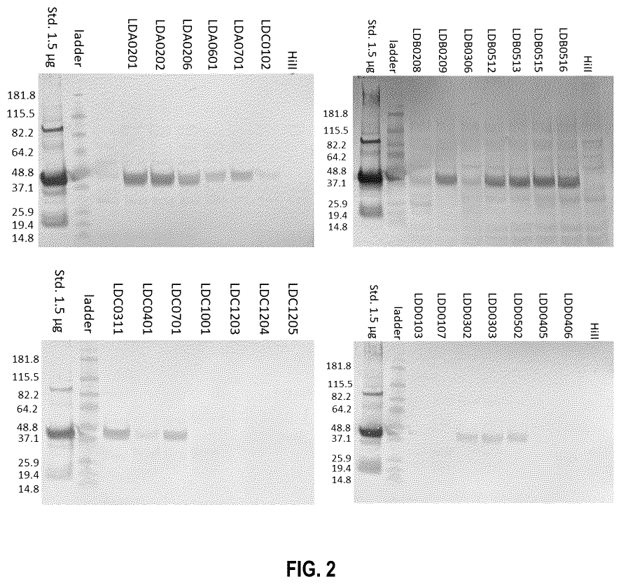

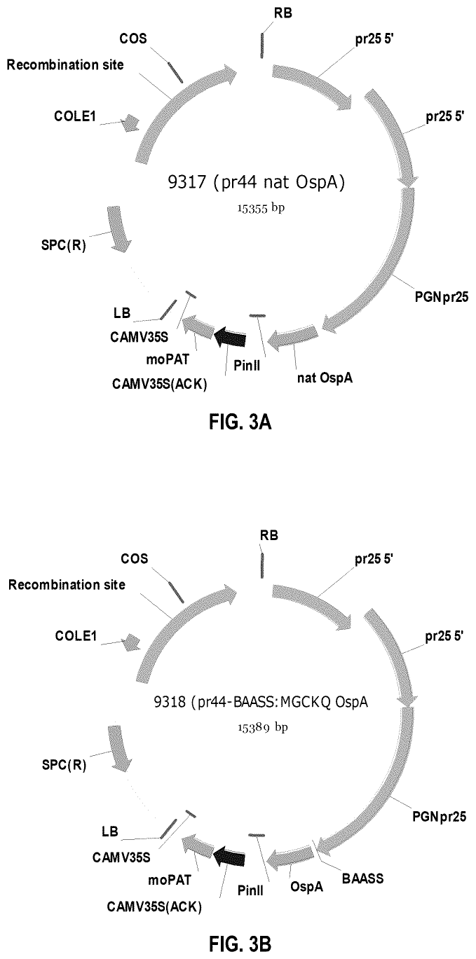

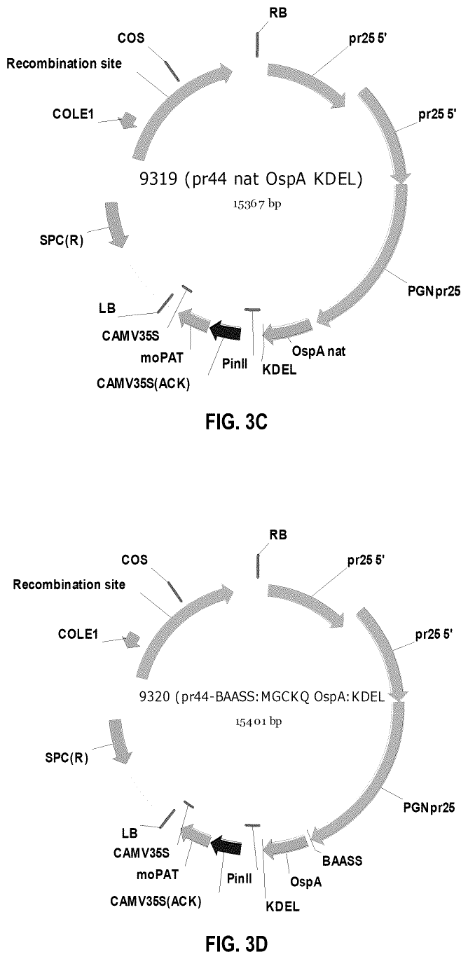

Western blot analysis showed substantial expression for all four constructs (FIG. 2) estimated at least 100 mg/kg up to 300 mg/kg whole seed. The western blots show a doublet band in some cases that may reflect post-translational modification.

These results demonstrated that the OspA outer antigen from Borrelia burgdorferi can be produced at high levels in transgenic maize.

### Example 2

Maize material was ground and mixed with control germ to obtain individual doses for mouse feeding. To begin to assess the efficacy of the maize-produced OspA in preventing infection with Borrelia burgdorferi, maize material is fed to mice and IgG immune response is measured.

### REFERENCES

(CDC), C. f. D. C. a. P. (2004) Lyme disease—United States, 2001-2002, MMWR Morb Mortal Wkly Rep 53, 365-369.
Adeolu, M., and Gupta, R. S. (2014) A phylogenomic and molecular marker based proposal for the division of the genus Borrelia into two genera: the emended genus Borrelia containing only the members of the relapsing fever Borrelia, and the genus Borreliella gen. nov. containing the members of the Lyme disease Borrelia (Borrelia burgdorferi sensu lato complex), Antonie Van Leeuwenhoek 105, 1049-1072.
Bailey, M. R. (2000) A model system for edible vaccination using recombinant avidin produced in corn seed, Texas A&M University.
Bockenstedt, L. K., Hodzic, E., Feng, S., Bourrel, K. W., de Silva, A., Montgomery, R. R., Fikrig, E., Radolf, J. D., and Barthold, S. W. (1997) Borrelia burgdorferi strain-specific Osp C-mediated immunity in mice, Infect Immun 65, 4661-4667.
Comstedt, P., Schuler, W., Meinke, A., and Lundberg, U. (2017) The novel Lyme borreliosis vaccine VLA15 shows broad protection against Borrelia species expressing six different OspA serotypes, PloS one 12, e0184357.
Daniell, H., Singh, N. D., Mason, H., and Streatfield, S. J. (2009) Plant-made vaccine antigens and biopharmaceuticals, Trends Plant Sci 14, 669-679.
de Silva A M, Z. N., Zhang Y, Dolan M C, Piesman J, Fikrig E. (1999) Influence of outer surface protein A antibody on Borrelia burgdorferi within feeding ticks., Infect Immun. 67, 30-35.
de Silva, A. M., Fish, D., Burkot, T. R., Zhang, Y., and Fikrig, E. (1997) OspA antibodies inhibit the acquisition of Borrelia burgdorferi by Ixodes ticks, Infect Immun 65, 3146-3150.
de Silva, A. M., Telford, S. R., 3rd, Brunet, L. R., Barthold, S. W., and Fikrig, E. (1996) Borrelia burgdorferi OspA is an arthropod-specific transmission-blocking Lyme disease vaccine, J Exp Med 183, 271-275.
Earnhart, C. G., and Marconi, R. T. (2007) An octavalent lyme disease vaccine induces antibodies that recognize all incorporated OspC type-specific sequences, Hum Vaccin 3, 281-289.
Earnhart, C. G., Buckles, E. L., and Marconi, R. T. (2007) Development of an OspC-based tetravalent, recombinant, chimeric vaccinogen that elicits bactericidal antibody against diverse Lyme disease spirochete strains, Vaccine 25, 466-480.
Earnhart, C. G., Buckles, E. L., Dumler, J. S., and Marconi, R. T. (2005) Demonstration of OspC type diversity in invasive human lyme disease isolates and identification of previously uncharacterized epitopes that define the specificity of the OspC murine antibody response, Infect Immun 73, 7869-7877.
Eschner, A. K., and Mugnai, K. (2015) Immunization with a recombinant subunit OspA vaccine markedly impacts the rate of newly acquired Borrelia burgdorferi infections in client-owned dogs living in a coastal community in Maine, USA, Parasit Vectors 8, 92.
Fikrig, E., Barthold, S. W., Kantor, F. S., and Flavell, R. A. (1990) Protection of mice against the Lyme disease agent by immunizing with recombinant OspA, Science 250, 553-556.
Fikrig, E., Barthold, S. W., Kantor, F. S., and Flavell, R. A. (1991) Protection of mice from Lyme borreliosis by oral vaccination with Escherichia coli expressing OspA, Journal of Infectious Diseases 164, 1224-1227.
Fikrig, E., Barthold, S. W., Kantor, F., and Flavell, R. (1992) Long-term protection of mice from Lyme disease by vaccination with OspA, Infection and immunity 60, 773-777.
Gomes-Solecki, M. J., Brisson, D. R., and Dattwyler, R. J. (2006) Oral vaccine that breaks the transmission cycle of the Lyme disease spirochete can be delivered via bait, Vaccine 24, 4440-4449.
Gomes-Solecki, M., Arnaboldi, P. M., Backenson, P. B., Benach, J. L., Cooper, C. L., Dattwyler, R. J., Diuk-Wasser, M., Fikrig, E., Hovius, J. W., Laegreid, W., Lundberg, U., Marconi, R. T., Marques, A. R., Molloy, P., Narasimhan, S., Pal, U., Pedra, J. H. F., Plotkin, S., Rock, D. L., Rosa, P., Telford, S. R., Tsao, J., Yang, X. F., and Schutzer, S. E. (2019) Protective Immunity and New Vaccines for Lyme Disease, Clin Infect Dis.
Grimm, D., Tilly, K., Byram, R., Stewart, P. E., Krum, J. G., Bueschel, D. M., Schwan, T. G., Policastro, P. F., Elias, A. F., and Rosa, P. A. (2004) Outer-surface protein C of the Lyme disease spirochete: a protein induced in ticks for infection of mammals, Proc Natl Acad Sci USA 101, 3142-3147.
Hayden, C. A., Egelkrout, E. M., Moscoso, A. M., Enrique, C., Keener, T. K., Jimenez-Flores, R., Wong, J. C., and Howard, J. A. (2012) Production of highly concentrated, heat-stable hepatitis B surface antigen in maize, Plant Biotechnology Journal 10, 979-984.
Hennig, A., Bonfig, K., Roitsch, T., and Warzecha, H. (2007) Expression of the recombinant bacterial outer surface protein A in tobacco chloroplasts leads to thylakoid localization and loss of photosynthesis, The FEBS journal 274, 5749-5758.
Hennig, A., Reinders, Y., Giritch, A., Reinders, J., and Warzecha, H. (2008) Assessment of different expression strategies for the production of a recombinant lipoprotein vaccine in plants, The Open Biotechnology Journal 2.
Hood, E. E., and Howard, J. A. (2014) Commercial plant-produced recombinant avidin, In Commercial Plant-Produced Recombinant Protein Products, pp 15-25, Springer.
Hood, E. E., Helmer, G. L., Fraley, R. T., and Chilton, M. D. (1986) The hypervirulence of Agrobacterium tumefaciens A281 is encoded in a region of pTiBo542 outside of T-DNA, J Bacteriol 168, 1291-1301.
Howard, J. A. (2005) Commercialization of biopharmaceutical and bioindustrial proteins from plants, Crop Science 45, 468-472.
Howard, J. A., and Hood, E. E. (2014) Commercial Plant-Produced Recombinant Protein Products, Springer.
Howard, J., and Hood, E. E. (2014) The Future of Plant-Produced Pharmaceuticals and Industrial Proteins, In Commercial Plant-Produced Recombinant Protein Products, pp 261-274, Springer.
Huang, N., Zhang, D., Johnson, B. J., and Macmanus, C. (2016) OspA Fusion Protein for Vaccination against Lyme Disease, Google Patents.
Ishida, Y., Saito, H., Ohta, S., Hiei, Y., Komari, T., and Kumashiro, T. (1996) High efficiency transformation of maize (Zea mays L.) mediated by Agrobacterium tumefaciens, Nat Biotechnol 14, 745-750.
Izac, J. R., O'Bier, N. S., Oliver, L. D., Jr., Camire, A. C., Earnhart, C. G., LeBlanc Rhodes, D. V., Young, B. F., Parnham, S. R., Davies, C., and Marconi, R. T. (2020) Development and optimization of OspC chimeritope vaccinogens for Lyme disease, Vaccine 38, 1915-1924.
Johnson, B. J., Sviat, S. L., Happ, C. M., Dunn, J. J., Frantz, J. C., Mayer, L. W., and Piesman, J. (1995) Incomplete protection of hamsters vaccinated with unlipidated OspA from Borrelia burgdorferi infection is associated with low levels of antibody to an epitope defined by mAb LA-2, Vaccine 13, 1086-1094.
Klempner, M. S., Hu, L. T., Evans, J., Schmid, C. H., Johnson, G. M., Trevino, R. P., Norton, D., Levy, L., Wall, D., McCall, J., Kosinski, M., and Weinstein, A. (2001) Two controlled trials of antibiotic treatment in patients with persistent symptoms and a history of Lyme disease, N Engl J Med 345, 85-92.
Komari, T., Hiei, Y., Saito, Y., Murai, N., and Kumashiro, T. (1996) Vectors carrying two separate T-DNAs for co-transformation of higher plants mediated by Agrobacterium tumefaciens and segregation of transformants free from selection markers, Plant Journal 10, 165-174.
Lamphear, B. J., Streatfield, S. J., Jilka, J. M., Brooks, C. A., Barker, D. K., Turner, D. D., Delaney, D. E., Garcia, M., Wiggins, B., Woodard, S. L., Hood, E. E., Tizard, I. R., Lawhorn, B., and Howard, J. A. (2002) Delivery of subunit vaccines in maize seed, J Control Release 85, 169-180.
Mason, H., and Herbst-Kralovetz, M. (2012) Plant-derived antigens as mucosal vaccines, In Mucosal Vaccines, pp 101-120, Springer.
Meirelles Richer, L., Aroso, M., Contente-Cuomo, T., Ivanova, L., and Gomes-Solecki, M. (2011) Reservoir targeted vaccine for lyme borreliosis induces a yearlong, neutralizing antibody response to OspA in white-footed mice, Clin Vaccine Immunol 18, 1809-1816.
Navarre, C., Delannoy, M., Lefebvre, B., Nader, J., Vanham, D., and Boutry, M. (2006) Expression and secretion of recombinant outer-surface protein A from the Lyme disease agent, Borrelia burgdorferi, in Nicotiana tabacum suspension cells, Transgenic research 15, 325.
Pal, U., Yang, X., Chen, M., Bockenstedt, L. K., Anderson, J. F., Flavell, R. A., Norgard, M. V., and Fikrig, E. (2004) OspC facilitates Borrelia burgdorferi invasion of Ixodes scapularis salivary glands, J Clin Invest 113, 220-230.
Richer, L. M., Brisson, D., Melo, R., Ostfeld, R. S., Zeidner, N., and Gomes-Solecki, M. (2014) Reservoir targeted vaccine against Borrelia burgdorferi: a new strategy to prevent Lyme disease transmission, J Infect Dis 209, 1972-1980.
Rybicki, E. P. (2009) Plant-produced vaccines: promise and reality, Drug Discovery Today 14, 16-24.
Scheckelhoff, M. R., Telford, S. R., and Hu, L. T. (2006) Protective efficacy of an oral vaccine to reduce carriage of Borrelia burgdorferi (strain N40) in mouse and tick reservoirs, Vaccine 24, 1949-1957.
Schwan, T. G., Piesman, J., Golde, W. T., Dolan, M. C., and Rosa, P. A. (1995) Induction of an outer surface protein on Borrelia burgdorferi during tick feeding, Proc Natl Acad Sci USA 92, 2909-2913.
Seinost, G., Golde, W. T., Berger, B. W., Dunn, J. J., Qiu, D., Dunkin, D. S., Dykhuizen, D. E., Luft, B. J., and Dattwyler, R. J. (1999) Infection with multiple strains of Borrelia burgdorferi sensu stricto in patients with Lyme disease, Arch Dermatol 135, 1329-1333.
Stafford, K. C., Williams, S. C., van Oosterwijk, J. G., Linske, M. A., Zatechka, S., Richer, L. M., Molaei, G., Przybyszewski, C., and Wikel, S. K. (2020) Field evaluation of a novel oral reservoir-targeted vaccine against Borrelia burgdorferi utilizing an inactivated whole-cell bacterial antigen expression vehicle, Experimental and Applied Acarology, 1-12.
Steere, A. C. (1989) Lyme disease [see comments], N Engl J Med 321, 586-596.
Steere, A. C., Sikand, V. K., Meurice, F., Parenti, D. L., Fikrig, E., Schoen, R. T., Nowakowski, J., Schmid, C. H., Laukamp, S., Buscarino, C., and Krause, D. S. (1998) Vaccination against Lyme disease with recombinant Borrelia burgdorferi outer-surface lipoprotein A with adjuvant. Lyme Disease Vaccine Study Group, N Engl J Med 339, 209-215.
Streatfield, S. J., and Howard, J. A. (2003) Plant production systems for vaccines.
Streatfield, S. J., Lane, J. R., Brooks, C. A., Barker, D. K., Poage, M. L., Mayor, J. M., Lamphear, B. J., Drees, C. F., Jilka, J. M., Hood, E. E., and Howard, J. A. (2003) Corn as a production system for human and animal vaccines, Vaccine 21, 812-815.
Tacket, C. O., Pasetti, M. F., Edelman, R., Howard, J. A., and Streatfield, S. (2004) Immunogenicity of recombinant LT-B delivered orally to humans in transgenic corn, Vaccine 22, 4385-4389.
Tsao, J., Barbour, A. G., Luke, C. J., Fikrig, E., and Fish, D. (2001) OspA immunization decreases transmission of Borrelia burgdorferi spirochetes from infected Peromyscus leucopus mice to larval Ixodes scapularis ticks, Vector Borne Zoonotic Dis 1, 65-74.
Wormser, G. P., Ramanathan, R., Nowakowski, J., McKenna, D., Holmgren, D., Visintainer, P., Dornbush, R., Singh, B., and Nadelman, R. B. (2003) Duration of antibiotic therapy for early Lyme disease. A randomized, double-blind, placebo-controlled trial, Ann Intern Med 138, 697-704.

SEQUENCES

SEQ ID NO: 1 - OspA coding sequence nucleotide (without signal sequence):
TGCAAGCAGAATGTTTCGTCGCTGGATGAGAAGAATAGCGTGTCCGTCGACCTCCCGGGCGA
GATGAAGGTGCTGGTCAGCAAGGAGAAGAACAAGGACGGCAAGTACGACCTCATCGCCACCG
TCGACAAGCTCGAGCTGAAGGGCACGTCCGACAAGAACAACGGCTCGGGCGTGCTGGAGGGC
GTCAAGGCGGACAAGTCCAAGGTCAAGCTGACCATCTCGGACGACCTCGGCCAGACCACCCT
GGAGGTGTTCAAGGAGGACGGCAAGACGCTGGTGTCCAAGAAGGTCACCAGCAAGGACAAGT
CCAGCACGGAGGAGAAGTTCAACGAGAAGGGCGAGGTGTCCGAGAAGATCATCACCAGGGCG
GACGGCACCAGGCTCGAGTACACCGGCATCAAGTCGGACGGCTCGGGCAAGGCTAAGGAGGT
GCTGAAGGGCTACGTCCTGGAGGGCACCCTGACCGCGGAGAAGACCACGCTCGTGGTCAAGG
AGGGCACCGTGACGCTGTCCAAGAACATCTCCAAGAGCGGCGAGGTGAGCGTCGAGCTCAAC
GACACCGACAGCTCGGCGGCCACCAAGAAGACGGCTGCCTGGAACTCGGGCACCAGCACGCT
CACCATCACGGTGAACAGCAAGAAGACGAAGGACCTGGTCTTCACCAAGGAGAACACCATCA
CGGTGCAGCAGTACGACTCGAACGGCACCAAGCTGGAGGGCTCGGCTGTGGAGATCACGAAG
CTGGACGAGATCAAGAACGCGCTCAAGTGATGA
SEQ ID NO: 2 - OspA protein (without signal sequence):
CKQNVSSLDEKNSVSVDLPGEMKVLVSKEKNKDGKYDLIATVDKLELKGTSDKNNGSGVLEG
VKADKSKVKLTISDDLGQTTLEVFKEDGKTLVSKKVTSKDKSSTEEKFNEKGEVSEKIITRA
DGTRLEYTGIKSDGSGKAKEVLKGYVLEGTLTAEKTTLVVKEGTVTLSKNISKSGEVSVELN
DTDSSAATKKTAAWNSGTSTLTITVNSKKTKDLVFTKENTITVQQYDSNGTKLEGSAVEITK
LDEIKNALK
SEQ ID NO: 3 -LDA coding sequence nucleotide:
ATGAAGAAGTACCTCCTGGGCATTGGGCTGATCCTCGCTCTGATTGCGTGCAAGCAGAATGT
TTCGTCGCTGGATGAGAAGAATAGCGTGTCCGTCGACCTCCCGGGCGAGATGAAGGTGCTGG
TCAGCAAGGAGAAGAACAAGGACGGCAAGTACGACCTCATCGCCACCGTCGACAAGCTCGAG
CTGAAGGGCACGTCCGACAAGAACAACGGCTCGGGCGTGCTGGAGGGCGTCAAGGCGGACAA
GTCCAAGGTCAAGCTGACCATCTCGGACGACCTCGGCCAGACCACCCTGGAGGTGTTCAAGG
AGGACGGCAAGACGCTGGTGTCCAAGAAGGTCACCAGCAAGGACAAGTCCAGCACGGAGGAG
AAGTTCAACGAGAAGGGCGAGGTGTCCGAGAAGATCATCACCAGGGCGGACGGCACCAGGCT
CGAGTACACCGGCATCAAGTCGGACGGCTCGGGCAAGGCTAAGGAGGTGCTGAAGGGCTACG
TCCTGGAGGGCACCCTGACCGCGGAGAAGACCACGCTCGTGGTCAAGGAGGGCACCGTGACG
CTGTCCAAGAACATCTCCAAGAGCGGCGAGGTGAGCGTCGAGCTCAACGACACCGACAGCTC
GGCGGCCACCAAGAAGACGGCTGCCTGGAACTCGGGCACCAGCACGCTCACCATCACGGTGA
ACAGCAAGAAGACGAAGGACCTGGTCTTCACCAAGGAGAACACCATCACGGTGCAGCAGTAC
GACTCGAACGGCACCAAGCTGGAGGGCTCGGCTGTGGAGATCACGAAGCTGGACGAGATCAA
GAACGCGCTCAAGTGATGA
SEQ ID NO: 4 - LDA protein:
MKKYLLGIGLILALIACKQNVSSLDEKNSVSVDLPGEMKVLVSKEKNKDGKYDLIATVDKLE
LKGTSDKNNGSGVLEGVKADKSKVKLTISDDLGQTTLEVFKEDGKTLVSKKVTSKDKSSTEE
KFNEKGEVSEKIITRADGTRLEYTGIKSDGSGKAKEVLKGYVLEGTLTAEKTTLVVKEGTVT
LSKNISKSGEVSVELNDTDSSAATKKTAAWNSGTSTLTITVNSKKTKDLVFTKENTITVQQY
DSNGTKLEGSAVEITKLDEIKNALK
SEQ ID NO: 5 - LDB coding sequence nucleotide:
CCATGGCCAATAAGCATCTCTCACTCTCACTGTTCCTCGTTCTCCTCGGGCTCTCCGCCTCG
CTCGCCTCTGGGGGCATGGGGTGCAAGCAGAACGTCTCCAGCCTCGACGAGAAGAACTCCGT
GAGCGTCGACCTCCCGGGCGAGATGAAGGTGCTCGTGTCCAAGGAGAAGAACAAGGACGGCA
AGTACGACCTGATCGCCACCGTGGACAAGCTCGAGCTGAAGGGCACGTCCGACAAGAACAAC
GGCTCGGGCGTGCTGGAGGGCGTCAAGGCGGACAAGTCCAAGGTCAAGCTGACCATCTCGGA
CGACCTCGGCCAGACCACCCTGGAGGTGTTCAAGGAGGACGGCAAGACGCTCGTGTCCAAGA
AGGTCACCAGCAAGGACAAGTCCAGCACGGAGGAGAAGTTCAACGAGAAGGGCGAGGTCAGC
GAGAAGATCATCACCAGGGCGGACGGCACCAGGCTCGAGTACACCGGCATCAAGTCGGACGG
CTCGGGCAAGGCTAAGGAGGTGCTGAAGGGCTACGTCCTGGAGGGCACCCTGACCGCGGAGA
AGACCACGCTCGTGGTCAAGGAGGGCACCGTGACGCTGTCCAAGAACATCTCCAAGAGCGGC
GAGGTGAGCGTCGAGCTGAACGACACCGACAGCTCGGCGGCCACCAAGAAGACGGCTGCCTG
GAACTCGGGCACCAGCACGCTCACCATCACGGTGAACTCCAAGAAGACGAAGGACCTGGTCT
TCACCAAGGAGAACACCATCACGGTGCAGCAGTACGACTCGAACGGCACCAAGCTGGAGGGC
TCGGCTGTCGAGATCACGAAGCTGGACGAGATCAAGAACGCGCTGAAGTAGTGA
SEQ ID NO: 6 - LDB protein:
MANKHLSLSLFLVLLGLSASLASGMGCKONVSSLDEKNSVSVDLPGEMKVLVSKEKNKDGKY
DLIATVDKLELKGTSDKNNGSGVLEGVKADKSKVKLTISDDLGOTTLEVFKEDGKTLVSKKV
TSKDKSSTEEKFNEKGEVSEKIITRADGTRLEYTGIKSDGSGKAKEVLKGYVLEGTLTAEKT
TLVVKEGTVTLSKNISKSGEVSVELNDTDSSAATKKTAAWNSGTSTLTITVNSKKTKDLVFT
KENTITVQQYDSNGTKLEGSAVEITKLDEIKNALK
SEQ ID NO: 7 - LDC coding sequence nucleotide:
ATGAAGAAGTACCTCCTGGGCATTGGGCTGATCCTCGCTCTGATTGCGTGCAAGCAGAATGT
TTCGTCGCTGGATGAGAAGAATAGCGTGTCCGTCGACCTCCCGGGCGAGATGAAGGTGCTGG
TCAGCAAGGAGAAGAACAAGGACGGCAAGTACGACCTCATCGCCACCGTCGACAAGCTCGAG
CTGAAGGGCACGTCCGACAAGAACAACGGCTCGGGCGTGCTGGAGGGCGTCAAGGCGGACAA
GTCCAAGGTCAAGCTGACCATCTCGGACGACCTCGGCCAGACCACCCTGGAGGTGTTCAAGG
AGGACGGCAAGACGCTGGTGTCCAAGAAGGTCACCAGCAAGGACAAGTCCAGCACGGAGGAG
AAGTTCAACGAGAAGGGCGAGGTGTCCGAGAAGATCATCACCAGGGCGGACGGCACCAGGCT
CGAGTACACCGGCATCAAGTCGGACGGCTCGGGCAAGGCTAAGGAGGTGCTGAAGGGCTACG
TCCTGGAGGGCACCCTGACCGCGGAGAAGACCACGCTCGTGGTCAAGGAGGGCACCGTGACG
CTGTCCAAGAACATCTCCAAGAGCGGCGAGGTGAGCGTCGAGCTCAACGACACCGACAGCTC
GGCGGCCACCAAGAAGACGGCTGCCTGGAACTCGGGCACCAGCACGCTCACCATCACGGTGA
ACAGCAAGAAGACGAAGGACCTGGTCTTCACCAAGGAGAACACCATCACGGTGCAGCAGTAC
GACTCGAACGGCACCAAGCTGGAGGGCTCGGCTGTGGAGATCACGAAGCTGGACGAGATCAA
GAACGCGCTCAAGAAGGACGAGCTGTGATGA
SEQ ID NO: 8 - LDC protein:
MKKYLLGIGLILALIACKQNVSSLDEKNSVSVDLPGEMKVLVSKEKNKDGKYDLIATVDKLE
LKGTSDKNNGSGVLEGVKADKSKVKLTISDDLGOTTLEVFKEDGKTLVSKKVTSKDKSSTEE
KFNEKGEVSEKIITRADGTRLEYTGIKSDGSGKAKEVLKGYVLEGTLTAEKTTLVVKEGTVT
LSKNISKSGEVSVELNDTDSSAATKKTAAWNSGTSTLTITVNSKKTKDLVFTKENTITVQQY
DSNGTKLEGSAVEITKLDEIKNALKDEL
SEQ ID NO: 9 - LDD coding sequence nucleotide:
CCATGGCCAATAAGCATCTCTCACTCTCACTGTTCCTCGTTCTCCTCGGGCTCTCCGCCTCG
CTCGCCTCTGGGGGCATGGGGTGCAAGCAGAACGTCTCCAGCCTCGACGAGAAGAACTCCGT
GAGCGTCGACCTCCCGGGCGAGATGAAGGTGCTCGTGTCCAAGGAGAAGAACAAGGACGGCA
AGTACGACCTGATCGCCACCGTGGACAAGCTCGAGCTGAAGGGCACGTCCGACAAGAACAAC
GGCTCGGGCGTGCTGGAGGGCGTCAAGGCGGACAAGTCCAAGGTCAAGCTGACCATCTCGGA
CGACCTCGGCCAGACCACCCTGGAGGTGTTCAAGGAGGACGGCAAGACGCTCGTGTCCAAGA
AGGTCACCAGCAAGGACAAGTCCAGCACGGAGGAGAAGTTCAACGAGAAGGGCGAGGTCAGC
GAGAAGATCATCACCAGGGCGGACGGCACCAGGCTCGAGTACACCGGCATCAAGTCGGACGG
CTCGGGCAAGGCTAAGGAGGTGCTGAAGGGCTACGTCCTGGAGGGCACCCTGACCGCGGAGA
AGACCACGCTCGTGGTCAAGGAGGGCACCGTGACGCTGTCCAAGAACATCTCCAAGAGCGGC
GAGGTGAGCGTCGAGCTGAACGACACCGACAGCTCGGCGGCCACCAAGAAGACGGCTGCCTG
GAACTCGGGCACCAGCACGCTCACCATCACGGTGAACTCCAAGAAGACGAAGGACCTGGTCT
TCACCAAGGAGAACACCATCACGGTGCAGCAGTACGACTCGAACGGCACCAAGCTGGAGGGC
TCGGCTGTCGAGATCACGAAGCTGGACGAGATCAAGAACGCGCTGAAGAAGGATGAGCTGTA
GTGA
SEQ ID NO: 10 - LDD protein:
MANKHLSLSLFLVLLGLSASLASGMGCKQNVSSLDEKNSVSVDLPGEMKVLVSKEKNKDGKY
DLIATVDKLELKGTSDKNNGSGVLEGVKADKSKVKLTISDDLGQTTLEVFKEDGKTLVSKKV
TSKDKSSTEEKFNEKGEVSEKIITRADGTRLEYTGIKSDGSGKAKEVLKGYVLEGTLTAEKT
TLVVKEGTVTLSKNISKSGEVSVELNDTDSSAATKKTAAWNSGTSTLTITVNSKKTKDLVFT
KENTITVQQYDSNGTKLEGSAVEITKLDEIKNALKDEL
SEQ ID NO: 11 - Pr44(3xpr25) - promoter nucleotide:
GGCGCGCCGGTATGAATTTGGAAACAAATTCAGTACTTTTAAAAAAATTTGTTGTAGGGAGC
AAATAATACATAAAATAATTTATGCATTATTTTATTTTTTATTTGTAATAATATGCTTGAAA
CGATAATTCAGTATGCATGTTGTGCCAGTGTACTACACGGGCGGGGGGAGGGGATTGAGTGG
GCCAGCGCGGTGCGTAGGGTAGATGGGCTGAAATTGATAACTCAAGTCCGACTAGGTTCTCT
TTTTATTTCCCTTCCTTTTCTATTTTCCTTTCTTTTAATTTTCATGCTTTCAAACTAAATTC
AAATTCGAGTTTTGAATTTCAGCTTCTAAATTGTACACTAAAATTATATGATAAGGTAACCC
CTACTATTACTTTTAATTTTTTTATTCTACCCCATATTGTTTACTTAGGGGAGAATAATTGA
CTTAATCACATTCTTCCTAGGTTTCAATTCTCAATCTTTCAAATCCACATTTTTAGATTTCT
ATTTTGAATTTAAATACCAGTTTGGATTTAGAGTTCAATTTCAAAATACACAACCAAAATAC
CAGCATGAATGCAAATATATTTTATGTTTATGTATTTACTTTTCTTTTATACTTTGCTCAAA
ATAGTTATTTTCATGTATGAAACTCAATAAGCAAGGAACTCACGTTATTATATAACCTAATA
GGAATAATTTAGGTAACATAATTTATCATCCTCTTGATTTAAAAGAGATATGCCTCCAGAAT
AAGACACATACTAAAAATAACTCTAATATTGAATAACTAAAGTCGTACAAATCTCTACTATT
ATTCCTATAAAATAATAAAGAACTAGCTACAACTTCTTTAAGGCATTATTCAGGGTTTACAG
CTTGAGAGGCATGAACCCATCCTGTATACTCCTGGACTTGGAAGACAAAATGTCAACCAAAG
TGAAAGGTTTTCTTATGGTTGCTGCTAAGAGATAGATTGAACACTAGATCTCTCCTAAGACG
TCAGGGCATGCGTTTAGACTCCTACACATGCGAAAACTGCATCTTACAGTTGGAAGAAACTA
TATCTCACCACTTCCTGCGGTGTAACTTTGCCCAAAGATGTTGGCTCACTGTTGGAATCACT
CCGCCCCGAACTTTGGATCTAACGCTTGCAGTGCTACATATTAGAGCAAGACTAACAATGCC
GTGGAGAATGGAAGGTATTATAACCATGTCATGGTGCATATGGAAATGTCGAAATAACTGGA
TATTCGAAAACATACCGCCAACGGTGGCGGCCTGCAAGGAAATGTTCAAGACTGAAATGAAC
TACATCTGCTACCAAGTTAAGCTCGAGACAGGAGCTAAAAGTAGAAACTGGATACAACACTT
TGTAACATAGTGACACTCCCCTTTTCCTTTCTTTTACCTTAGAACTATACATACAATCCACA
TTCAATAAAAATTTGTAGGTACGCCATACACACTACCGGAATCCGGCTCTTTGCCGAGTGTG
AGGCGCTTTGTCGAGTGCTTTTTGTCCAGCACTCGGCAAAAAAGTCTTTGCCATGTGCCGCA
CTCGGCAAAGTCCTGCTCTCGGTAACGACCGCGTTTACCGAGAGCAGGACTCTCGACACAGA
AATACACTCGACAAAGAAATCTTTGCCGAGAGCCAAACACTCGGCGAACGGCAGCGCTCGGC
AAAGGGTCGTCAGCCGCCGTCTAAAGCTGACGGTCGTTATCTTTGTCGAGTGCCCCCTCGTC
CGACACTCAGTAGAGCACGCGCCGGTATGAATTTGGAAACAAATTCAGTACTTTTAAAAAAA
TTTGTTGTAGGGAGCAAATAATACATAAAATAATTTATGCATTATTTTATTTTTTATTTGTA
ATAATATGCTTGAAACGATAATTCAGTATGCATGTTGTGCCAGTGTACTACACGGGCGGGGG
GAGGGGATTGAGTGGGCCAGCGCGGTGCGTAGGGTAGATGGGCTGAAATTGATAACTCAAGT
CCGACTAGGTTCTCTTTTTATTTCCCTTCCTTTTCTATTTTCCTTTCTTTTAATTTTCATGC
TTTCAAACTAAATTCAAATTCGAGTTTTGAATTTCAGCTTCTAAATTGTACACTAAAATTAT
ATGATAAGGTAACCCCTACTATTACTTTTAATTTTTTTATTCTACCCCATATTGTTTACTTA
GGGGAGAATAATTGACTTAATCACATTCTTCCTAGGTTTCAATTCTCAATCTTTCAAATCCA
CATTTTTAGATTTCTATTTTGAATTTAAATACCAGTTTGGATTTAGAGTTCAATTTCAAAAT
ACACAACCAAAATACCAGCATGAATGCAAATATATTTTATGTTTATGTATTTACTTTTCTTT
TATACTTTGCTCAAAATAGTTATTTTCATGTATGAAACTCAATAAGCAAGGAACTCACGTTA
TTATATAACCTAATAGGAATAATTTAGGTAACATAATTTATCATCCTCTTGATTTAAAAGAG
ATATGCCTCCAGAATAAGACACATACTAAAAATAACTCTAATATTGAATAACTAAAGTCGTA
CAAATCTCTACTATTATTCCTATAAAATAATAAAGAACTAGCTACAACTTCTTTAAGGCATT
ATTCAGGGTTTACAGCTTGAGAGGCATGAACCCATCCTGTATACTCCTGGACTTGGAAGACA
AAATGTCAACCAAAGTGAAAGGTTTTCTTATGGTTGCTGCTAAGAGATAGATTGAACACTAG
ATCTCTCCTAAGACGTCAGGGCATGCGTTTAGACTCCTACACATGCGAAAACTGCATCTTAC
AGTTGGAAGAAACTATATCTCACCACTTCCTGCGGTGTAACTTTGCCCAAAGATGTTGGCTC
ACTGTTGGAATCACTCCGCCCCGAACTTTGGATCTAACGCTTGCAGTGCTACATATTAGAGC
AAGACTAACAATGCCGTGGAGAATGGAAGGTATTATAACCATGTCATGGTGCATATGGAAAT
GTCGAAATAACTGGATATTCGAAAACATACCGCCAACGGTGGCGGCCTGCAAGGAAATGTTC
AAGACTGAAATGAACTACATCTGCTACCAAGTTAAGCTCGAGACAGGAGCTAAAAGTAGAAA
CTGGATACAACACTTTGTAACATAGTGACACTCCCCTTTTCCTTTCTTTTACCTTAGAACTA
TACATACAATCCACATTCAATAAAAATTTGTAGGTACGCCATACACACTACCGGAATCCGGC
TCTTTGCCGAGTGTGAGGCGCTTTGTCGAGTGCTTTTTGTCCAGCACTCGGCAAAAAAGTCT
TTGCCATGTGCCGCACTCGGCAAAGTCCTGCTCTCGGTAACGACCGCGTTTACCGAGAGCAG
GACTCTCGACACAGAAATACACTCGACAAAGAAATCTTTGCCGAGAGCCAAACACTCGGCGA
ACGGCAGCGCTCGGCAAAGGGTCGTCAGCCGCCGTCTAAAGCTGACGGTCGTTATCTTTGTC
GAGTGCCCCCTCGTCCGACACTCAGTAGAGCACGCGCCGGTATGAATTTGGAAACAAATTCA
GTACTTTTAAAAAAATTTGTTGTAGGGAGCAAATAATACATAAAATAATTTATGCATTATTT
TATTTTTTATTTGTAATAATATGCTTGAAACGATAATTCAGTATGCATGTTGTGCCAGTGTA
CTACACGGGCGGGGGGAGGGGATTGAGTGGGCCAGCGCGGTGCGTAGGGTAGATGGGCTGAA
ATTGATAACTCAAGTCCGACTAGGTTCTCTTTTTATTTCCCTTCCTTTTCTATTTTCCTTTC
TTTTAATTTTCATGCTTTCAAACTAAATTCAAATTCGAGTTTTGAATTTCAGCTTCTAAATT
GTACACTAAAATTATATGATAAGGTAACCCCTACTATTACTTTTAATTTTTTTATTCTACCC
CATATTGTTTACTTAGGGGAGAATAATTGACTTAATCACATTCTTCCTAGGTTTCAATTCTC
AATCTTTCAAATCCACATTTTTAGATTTCTATTTTGAATTTAAATACCAGTTTGGATTTAGA
GTTCAATTTCAAAATACACAACCAAAATACCAGCATGAATGCAAATATATTTTATGTTTATG
TATTTACTTTTCTTTTATACTTTGCTCAAAATAGTTATTTTCATGTATGAAACTCAATAAGC
AAGGAACTCACGTTATTATATAACCTAATAGGAATAATTTAGGTAACATAATTTATCATCCT
CTTGATTTAAAAGAGATATGCCTCCAGAATAAGACACATACTAAAAATAACTCTAATATTGA
ATAACTAAAGTCGTACAAATCTCTACTATTATTCCTATAAAATAATAAAGAACTAGCTACAA
CTTCTTTAAGGCATTATTCAGGGTTTACAGCTTGAGAGGCATGAACCCATCCTGTATACTCC
TGGACTTGGAAGACAAAATGTCAACCAAAGTGAAAGGTTTTCTTATGGTTGCTGCTAAGAGA
TAGATTGAACACTAGATCTCTCCTAAGACGTCAGGGCATGCGTTTAGACTCCTACACATGCG
AAAACTGCATCTTACAGTTGGAAGAAACTATATCTCACCACTTCCTGCGGTGTAACTTTGCC
CAAAGATGTTGGCTCACTGTTGGAATCACTCCGCCCCGAACTTTGGATCTAACGCTTGCAGT
GCTACATATTAGAGCAAGACTAACAATGCCGTGGAGAATGGAAGGTATTATAACCATGTCAT
GGTGCATATGGAAATGTCGAAATAACTGGATATTCGAAAACATACCGCCAACGGTGGCGGCC
TGCAAGGAAATGTTCAAGACTGAAATGAACTACATCTGCTACCAAGTTAAGCTCGAGACAGG
AGCTAAAAGTAGAAACTGGATACAACACTTTGTAACATAGTGACACTCCCCTTTTCCTTTCT
TTTACCTTAGAACTATACATACAATCCACATTCAATAAAAATTTGTAGGTACGCCATACACA
CTACCGGAATCCGGCTCTTTGCCGAGTGTGAGGCGCTTTGTCGAGTGCTTTTTGTCCAGCAC
TCGGCAAAAAAGTCTTTGCCATGTGCCGCACTCGGCAAAGTCCTGCTCTCGGTAACGACCGC
GTTTACCGAGAGCAGGACTCTCGACACAGAAATACACTCGACAAAGAAATCTTTGCCGAGAG
CCAAACACTCGGCGAACGGCAGCGCTCGGCAAAGGGTCGTCAGCCGCCGTCTAAAGCTGACG
GTCGTTATCTTTGTCGAGTGCCCCCTCGTCCGACACTCAGTAGAGCAAGCTTGCCGAGTGCC
ATCCTTGGACACTCGATAAAGTATATTTTATTTTTTTTTATTTTGCCAACCAAACTTTTTGT
GGTATGTTCCTACACTATGTAGATCTACATGTACCATTTTGGCACAATTACAAAAATGTTTT
CTATAACTATTAGATTTAGTTCGTTTATTTGAATTTCTTCGGAAAATTCACATATGAACTGC
AAGTCACTCGAAACATGAAAAACCGTGCATGCAAAATAAATGATATGCATGTTATCTAGCAC
AAGTTACGACCGAATTCAGAAGCAGACCAGAATCTTCAAGCACCATGCTCACTAAACATGAC
CGTGAACTTGTTATCCAGTTGTTTAAAAATTGTATAAAACACAAATAAAGTCAGAAATTAAT
GAAACTTGTCCACATGTCATGATATCATATATAGAGGTTGTGATAAAAATTTGATAATGTTT
CGGTAAAGTTGTGACGTACTATGTGTAGAAACCTAAGTGACCTACACATAAAATCATAGAGT
TTCAATGTAGTTCACTCGACAAAGACTTTGTCAAGTGTCCGATAAAAAGTATTCAGCAAAGA
AGCCGTTGTCGATTTACTGTTCGTCGAGATCTCTTTGCCGAGTGTCACACTAGGCAAAGTCT
TTACGGAGTGTTTTTCAGGCTTTGACACTCGGCAAAGCGCTCGATTCCAGTAGTGACAGTAA
TTTGCATCAAAAATAGCCGAGAGATTTAAAATGAGTCAACTAATAGACCAACTAATTATTAG
CTATTAGTCGTTAGCTTCTTTAATCTAAGCTAAAACCAACTAATAGCTTATTTGTTGAATTA
CAATTAGCTCAACGGAATTCTCTGTTTTTTCTATAAAAAAAAGGGAAACTGCCCCTCATTTA
CAGCAAACTGTCCGCTGCCTGTCGTCCAGATACAATGAACGTACCTAGTAGGAACTCTTTTA
CACGCTCGGTCGCTCGCCGCGGATCGGAGTCCCAGGAACACGACACCACTGTGGAACACGAC
AAAGTCTGCTCAGAGGCGGCCACACCCTGGCGTGCACCGAGCCGGAGCCCGGATAAGCACGG
TAAGGAGAGTACGGCGGGACGTGGCGACCCGTGTGTCTGCTGCCACGCAGCCTTCCTCCACG
TAGCCGCGCGGCCGCGCCACGTACCAGGGCCCGGCGCTGGTATAAATGCGCGCCACCTCCGC
TTTAGTTCTGCATACAGCCAACCCAACACACACCCGAGCATATCACAGTGACAGACACTACA
SEQ ID NO: 12 - PinII -terminator nucleotide:
CTAGACTTGTCCATCTTCTGGATTGGCCAACTTAATTAATGTATGAAATAAAAGGATGCACA
CATAGTGACATGCTAATCACTATAATGTGGGCATCAAAGTTGTGTGTTATGTGTAATTACTA
GTTATCTGAATAAAAGAGAAAGAGATCATCCATATTTCTTATCCTAAATGAATGTCACGTGT
CTTTATAATTCTTTGATGAACCAGATGCATTTCATTAACCAAATCCATATACATATAAATAT
TAATCATATATAATTAATATCAATTGGGTTAGCAAAACAAATCTAGTCTAGGTGTGTTTTGC
GAAT

What is claimed is:

1. A method of producing a protective response to Borrelia burgdorferi in an animal, the method comprising:
administering to the animal a composition comprising a plant or plant product comprising the OspA polypeptide of Borrelia burgdorferi, or a sequence having at least 90% sequence identity thereto or a functional fragment thereof; and
producing a protective response to Borrelia burgdorferi in the animal.

2. The method of claim 1, wherein said administration is orally.

3. The method of claim 1, wherein the composition comprises seed or embryo of seed.

4. The method of claim 1, wherein the OspA polypeptide is expressed at levels of at least 10 mg/kg in seed of the plant.

5. The method of claim 1, wherein the protective response comprises a serum antibody response in the animal.

6. The method of claim 5, wherein the serum antibody response is at least 4 times greater than serum antibody response in an animal not administered the composition.

7. The method of claim 1, wherein the OspA polypeptide encoding sequence is optimized for maize expression.

8. The method of claim 1, wherein the OspA polypeptide is targeted to the apoplast or to the endoplasmic reticulum.

9. A vaccine comprising a plant-produced OspA polypeptide of Borrelia burgdorferi, the vaccine comprising a plant or plant product comprising a construct comprising:
(a) a promoter preferentially directing expression to seed of a plant;
(b) a nucleic acid molecule encoding the OspA polypeptide of Borrelia burgdorferi, or a sequence having at least 90% sequence identity thereto or a functional fragment operably linked to the promoter; and
(c) a nucleic acid molecule targeting expression of the polypeptide to the apoplast or endoplasmic reticulum of the plant.

10. The vaccine of claim 9, wherein the plant product comprises seed or embryo of seed.

11. The vaccine of claim 9, wherein the OspA polypeptide is expressed at levels of at least 10 mg/kg in seed of the plant.

12. The vaccine of claim 9, wherein the OspA polypeptide encoding sequence is optimized for maize expression.

13. The vaccine of claim 9, wherein the construct comprises SEQ ID NO: 1, 3, 5, 7, or 9, or a sequence with at least 90% sequence identity thereto.

14. The vaccine of claim 9, wherein the construct encodes SEQ ID NO: 2, 4, 6, 8, or 10, or a sequence with at least 90% sequence identity thereto.

15. A method of expressing the OspA polypeptide of Borrelia burgdorferi or a functional fragment thereof, the method comprising introducing into a plant a construct comprising:
(a) a promoter preferentially directing expression to seed of a plant;
(b) a nucleic acid molecule encoding the OspA polypeptide of Borrelia burgdorferi, or a sequence having at least 90% sequence identity thereto or a functional fragment operably linked to the promoter; and
(c) a nucleic acid molecule targeting expression of the polypeptide to the apoplast or endoplasmic reticulum of the plant.

16. The method of claim 15, wherein the OspA polypeptide is expressed at levels of at least 10 mg/kg in seed of the plant.

17. The method of claim 15, wherein the OspA polypeptide encoding sequence is optimized for maize expression.

18. The method of claim 15, wherein the construct comprises SEQ ID NO: 1, 3, 5, 7, or 9, or a sequence with at least 90% sequence identity thereto.

19. The method of claim 15, wherein the construct encodes SEQ ID NO: 2, 4, 6, 8, or 10, or a sequence with at least 90% sequence identity thereto.


SEQUENCE LISTING (12 sequences)

SEQ ID NO: 1 - DNA, 777 bp, synthetic construct
  source 1..777: mol_type = other DNA
tgcaagcagaatgtttcgtcgctggatgagaagaatagcgtgtccgtcgacctcccgggcgagatgaaggtgctggtcagcaaggagaagaacaaggacggcaagtacgacctcatcgccaccgtcgacaagctcgagctgaagggcacgtccgacaagaacaacggctcgggcgtgctggagggcgtcaaggcggacaagtccaaggtcaagctgaccatctcggacgacctcggccagaccaccctggaggtgttcaaggaggacggcaagacgctggtgtccaagaaggtcaccagcaaggacaagtccagcacggaggagaagttcaacgagaagggcgaggtgtccgagaagatcatcaccagggcggacggcaccaggctcgagtacaccggcatcaagtcggacggctcgggcaaggctaaggaggtgctgaagggctacgtcctggagggcaccctgaccgcggagaagaccacgctcgtggtcaaggagggcaccgtgacgctgtccaagaacatctccaagagcggcgaggtgagcgtcgagctcaacgacaccgacagctcggcggccaccaagaagacggctgcctggaactcgggcaccagcacgctcaccatcacggtgaacagcaagaagacgaaggacctggtcttcaccaaggagaacaccatcacggtgcagcagtacgactcgaacggcaccaagctggagggctcggctgtggagatcacgaagctggacgagatcaagaacgcgctcaagtgatga

SEQ ID NO: 2 - protein, 257 aa, synthetic construct
CKQNVSSLDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYVLEGTLTAEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 3 - DNA, 825 bp, synthetic construct
  source 1..825: mol_type = other DNA
atgaagaagtacctcctgggcattgggctgatcctcgctctgattgcgtgcaagcagaatgtttcgtcgctggatgagaagaatagcgtgtccgtcgacctcccgggcgagatgaaggtgctggtcagcaaggagaagaacaaggacggcaagtacgacctcatcgccaccgtcgacaagctcgagctgaagggcacgtccgacaagaacaacggctcgggcgtgctggagggcgtcaaggcggacaagtccaaggtcaagctgaccatctcggacgacctcggccagaccaccctggaggtgttcaaggaggacggcaagacgctggtgtccaagaaggtcaccagcaaggacaagtccagcacggaggagaagttcaacgagaagggcgaggtgtccgagaagatcatcaccagggcggacggcaccaggctcgagtacaccggcatcaagtcggacggctcgggcaaggctaaggaggtgctgaagggctacgtcctggagggcaccctgaccgcggagaagaccacgctcgtggtcaaggagggcaccgtgacgctgtccaagaacatctccaagagcggcgaggtgagcgtcgagctcaacgacaccgacagctcggcggccaccaagaagacggctgcctggaactcgggcaccagcacgctcaccatcacggtgaacagcaagaagacgaaggacctggtcttcaccaaggagaacaccatcacggtgcagcagtacgactcgaacggcaccaagctggagggctcggctgtggagatcacgaagctggacgagatcaagaacgcgctcaagtgatga

SEQ ID NO: 4 - protein, 273 aa, synthetic construct
MKKYLLGIGLILALIACKQNVSSLDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYVLEGTLTAEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 5 - DNA, 860 bp, synthetic construct
  source 1..860: mol_type = other DNA
ccatggccaataagcatctctcactctcactgttcctcgttctcctcgggctctccgcctcgctcgcctctgggggcatggggtgcaagcagaacgtctccagcctcgacgagaagaactccgtgagcgtcgacctcccgggcgagatgaaggtgctcgtgtccaaggagaagaacaaggacggcaagtacgacctgatcgccaccgtggacaagctcgagctgaagggcacgtccgacaagaacaacggctcgggcgtgctggagggcgtcaaggcggacaagtccaaggtcaagctgaccatctcggacgacctcggccagaccaccctggaggtgttcaaggaggacggcaagacgctcgtgtccaagaaggtcaccagcaaggacaagtccagcacggaggagaagttcaacgagaagggcgaggtcagcgagaagatcatcaccagggcggacggcaccaggctcgagtacaccggcatcaagtcggacggctcgggcaaggctaaggaggtgctgaagggctacgtcctggagggcaccctgaccgcggagaagaccacgctcgtggtcaaggagggcaccgtgacgctgtccaagaacatctccaagagcggcgaggtgagcgtcgagctgaacgacaccgacagctcggcggccaccaagaagacggctgcctggaactcgggcaccagcacgctcaccatcacggtgaactccaagaagacgaaggacctggtcttcaccaaggagaacaccatcacggtgcagcagtacgactcgaacggcaccaagctggagggctcggctgtcgagatcacgaagctggacgagatcaagaacgcgctgaagtagtga

SEQ ID NO: 6 - protein, 283 aa, synthetic construct
MANKHLSLSLFLVLLGLSASLASGMGCKQNVSSLDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYVLEGTLTAEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 7 - DNA, 837 bp, synthetic construct
  source 1..837: mol_type = other DNA
atgaagaagtacctcctgggcattgggctgatcctcgctctgattgcgtgcaagcagaatgtttcgtcgctggatgagaagaatagcgtgtccgtcgacctcccgggcgagatgaaggtgctggtcagcaaggagaagaacaaggacggcaagtacgacctcatcgccaccgtcgacaagctcgagctgaagggcacgtccgacaagaacaacggctcgggcgtgctggagggcgtcaaggcggacaagtccaaggtcaagctgaccatctcggacgacctcggccagaccaccctggaggtgttcaaggaggacggcaagacgctggtgtccaagaaggtcaccagcaaggacaagtccagcacggaggagaagttcaacgagaagggcgaggtgtccgagaagatcatcaccagggcggacggcaccaggctcgagtacaccggcatcaagtcggacggctcgggcaaggctaaggaggtgctgaagggctacgtcctggagggcaccctgaccgcggagaagaccacgctcgtggtcaaggagggcaccgtgacgctgtccaagaacatctccaagagcggcgaggtgagcgtcgagctcaacgacaccgacagctcggcggccaccaagaagacggctgcctggaactcgggcaccagcacgctcaccatcacggtgaacagcaagaagacgaaggacctggtcttcaccaaggagaacaccatcacggtgcagcagtacgactcgaacggcaccaagctggagggctcggctgtggagatcacgaagctggacgagatcaagaacgcgctcaagaaggacgagctgtgatga

SEQ ID NO: 8 - protein, 276 aa, synthetic construct
MKKYLLGIGLILALIACKQNVSSLDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYVLEGTLTAEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKDEL

SEQ ID NO: 9 - DNA, 872 bp, synthetic construct
  source 1..872: mol_type = other DNA
ccatggccaataagcatctctcactctcactgttcctcgttctcctcgggctctccgcctcgctcgcctctgggggcatggggtgcaagcagaacgtctccagcctcgacgagaagaactccgtgagcgtcgacctcccgggcgagatgaaggtgctcgtgtccaaggagaagaacaaggacggcaagtacgacctgatcgccaccgtggacaagctcgagctgaagggcacgtccgacaagaacaacggctcgggcgtgctggagggcgtcaaggcggacaagtccaaggtcaagctgaccatctcggacgacctcggccagaccaccctggaggtgttcaaggaggacggcaagacgctcgtgtccaagaaggtcaccagcaaggacaagtccagcacggaggagaagttcaacgagaagggcgaggtcagcgagaagatcatcaccagggcggacggcaccaggctcgagtacaccggcatcaagtcggacggctcgggcaaggctaaggaggtgctgaagggctacgtcctggagggcaccctgaccgcggagaagaccacgctcgtggtcaaggagggcaccgtgacgctgtccaagaacatctccaagagcggcgaggtgagcgtcgagctgaacgacaccgacagctcggcggccaccaagaagacggctgcctggaactcgggcaccagcacgctcaccatcacggtgaactccaagaagacgaaggacctggtcttcaccaaggagaacaccatcacggtgcagcagtacgactcgaacggcaccaagctggagggctcggctgtcgagatcacgaagctggacgagatcaagaacgcgctgaagaaggatgagctgtagtga

SEQ ID NO: 10 - protein, 286 aa, synthetic construct
MANKHLSLSLFLVLLGLSASLASGMGCKQNVSSLDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYVLEGTLTAEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALKDEL

SEQ ID NO: 11 - DNA, 6510 bp, synthetic construct
  source 1..6510: mol_type = other DNA
ggcgcgccggtatgaatttggaaacaaattcagtacttttaaaaaaatttgttgtagggagcaaataatacataaaataatttatgcattattttattttttatttgtaataatatgcttgaaacgataattcagtatgcatgttgtgccagtgtactacacgggcggggggaggggattgagtgggccagcgcggtgcgtagggtagatgggctgaaattgataactcaagtccgactaggttctctttttatttcccttccttttctattttcctttcttttaattttcatgctttcaaactaaattcaaattcgagttttgaatttcagcttctaaattgtacactaaaattatatgataaggtaacccctactattacttttaatttttttattctaccccatattgtttacttaggggagaataattgacttaatcacattcttcctaggtttcaattctcaatctttcaaatccacatttttagatttctattttgaatttaaataccagtttggatttagagttcaatttcaaaatacacaaccaaaataccagcatgaatgcaaatatattttatgtttatgtatttacttttcttttatactttgctcaaaatagttattttcatgtatgaaactcaataagcaaggaactcacgttattatataacctaataggaataatttaggtaacataatttatcatcctcttgatttaaaagagatatgcctccagaataagacacatactaaaaataactctaatattgaataactaaagtcgtacaaatctctactattattcctataaaataataaagaactagctacaacttctttaaggcattattcagggtttacagcttgagaggcatgaacccatcctgtatactcctggacttggaagacaaaatgtcaaccaaagtgaaaggttttcttatggttgctgctaagagatagattgaacactagatctctcctaagacgtcagggcatgcgtttagactcctacacatgcgaaaactgcatcttacagttggaagaaactatatctcaccacttcctgcggtgtaactttgcccaaagatgttggctcactgttggaatcactccgccccgaactttggatctaacgcttgcagtgctacatattagagcaagactaacaatgccgtggagaatggaaggtattataaccatgtcatggtgcatatggaaatgtcgaaataactggatattcgaaaacataccgccaacggtggcggcctgcaaggaaatgttcaagactgaaatgaactacatctgctaccaagttaagctcgagacaggagctaaaagtagaaactggatacaacactttgtaacatagtgacactccccttttcctttcttttaccttagaactatacatacaatccacattcaataaaaatttgtaggtacgccatacacactaccggaatccggctctttgccgagtgtgaggcgctttgtcgagtgctttttgtccagcactcggcaaaaaagtctttgccatgtgccgcactcggcaaagtcctgctctcggtaacgaccgcgtttaccgagagcaggactctcgacacagaaatacactcgacaaagaaatctttgccgagagccaaacactcggcgaacggcagcgctcggcaaagggtcgtcagccgccgtctaaagctgacggtcgttatctttgtcgagtgccccctcgtccgacactcagtagagcacgcgccggtatgaatttggaaacaaattcagtacttttaaaaaaatttgttgtagggagcaaataatacataaaataatttatgcattattttattttttatttgtaataatatgcttgaaacgataattcagtatgcatgttgtgccagtgtactacacgggcggggggaggggattgagtgggccagcgcggtgcgtagggtagatgggctgaaattgataactcaagtccgactaggttctctttttatttcccttccttttctattttcctttcttttaattttcatgctttcaaactaaattcaaattcgagttttgaatttcagcttctaaattgtacactaaaattatatgataaggtaacccctactattacttttaatttttttattctaccccatattgtttacttaggggagaataattgacttaatcacattcttcctaggtttcaattctcaatctttcaaatccacatttttagatttctattttgaatttaaataccagtttggatttagagttcaatttcaaaatacacaaccaaaataccagcatgaatgcaaatatattttatgtttatgtatttacttttcttttatactttgctcaaaatagttattttcatgtatgaaactcaataagcaaggaactcacgttattatataacctaataggaataatttaggtaacataatttatcatcctcttgatttaaaagagatatgcctccagaataagacacatactaaaaataactctaatattgaataactaaagtcgtacaaatctctactattattcctataaaataataaagaactagctacaacttctttaaggcattattcagggtttacagcttgagaggcatgaacccatcctgtatactcctggacttggaagacaaaatgtcaaccaaagtgaaaggttttcttatggttgctgctaagagatagattgaacactagatctctcctaagacgtcagggcatgcgtttagactcctacacatgcgaaaactgcatcttacagttggaagaaactatatctcaccacttcctgcggtgtaactttgcccaaagatgttggctcactgttggaatcactccgccccgaactttggatctaacgcttgcagtgctacatattagagcaagactaacaatgccgtggagaatggaaggtattataaccatgtcatggtgcatatggaaatgtcgaaataactggatattcgaaaacataccgccaacggtggcggcctgcaaggaaatgttcaagactgaaatgaactacatctgctaccaagttaagctcgagacaggagctaaaagtagaaactggatacaacactttgtaacatagtgacactccccttttcctttcttttaccttagaactatacatacaatccacattcaataaaaatttgtaggtacgccatacacactaccggaatccggctctttgccgagtgtgaggcgctttgtcgagtgctttttgtccagcactcggcaaaaaagtctttgccatgtgccgcactcggcaaagtcctgctctcggtaacgaccgcgtttaccgagagcaggactctcgacacagaaatacactcgacaaagaaatctttgccgagagccaaacactcggcgaacggcagcgctcggcaaagggtcgtcagccgccgtctaaagctgacggtcgttatctttgtcgagtgccccctcgtccgacactcagtagagcacgcgccggtatgaatttggaaacaaattcagtacttttaaaaaaatttgttgtagggagcaaataatacataaaataatttatgcattattttattttttatttgtaataatatgcttgaaacgataattcagtatgcatgttgtgccagtgtactacacgggcggggggaggggattgagtgggccagcgcggtgcgtagggtagatgggctgaaattgataactcaagtccgactaggttctctttttatttcccttccttttctattttcctttcttttaattttcatgctttcaaactaaattcaaattcgagttttgaatttcagcttctaaattgtacactaaaattatatgataaggtaacccctactattacttttaatttttttattctaccccatattgtttacttaggggagaataattgacttaatcacattcttcctaggtttcaattctcaatctttcaaatccacatttttagatttctattttgaatttaaataccagtttggatttagagttcaatttcaaaatacacaaccaaaataccagcatgaatgcaaatatattttatgtttatgtatttacttttcttttatactttgctcaaaatagttattttcatgtatgaaactcaataagcaaggaactcacgttattatataacctaataggaataatttaggtaacataatttatcatcctcttgatttaaaagagatatgcctccagaataagacacatactaaaaataactctaatattgaataactaaagtcgtacaaatctctactattattcctataaaataataaagaactagctacaacttctttaaggcattattcagggtttacagcttgagaggcatgaacccatcctgtatactcctggacttggaagacaaaatgtcaaccaaagtgaaaggttttcttatggttgctgctaagagatagattgaacactagatctctcctaagacgtcagggcatgcgtttagactcctacacatgcgaaaactgcatcttacagttggaagaaactatatctcaccacttcctgcggtgtaactttgcccaaagatgttggctcactgttggaatcactccgccccgaactttggatctaacgcttgcagtgctacatattagagcaagactaacaatgccgtggagaatggaaggtattataaccatgtcatggtgcatatggaaatgtcgaaataactggatattcgaaaacataccgccaacggtggcggcctgcaaggaaatgttcaagactgaaatgaactacatctgctaccaagttaagctcgagacaggagctaaaagtagaaactggatacaacactttgtaacatagtgacactccccttttcctttcttttaccttagaactatacatacaatccacattcaataaaaatttgtaggtacgccatacacactaccggaatccggctctttgccgagtgtgaggcgctttgtcgagtgctttttgtccagcactcggcaaaaaagtctttgccatgtgccgcactcggcaaagtcctgctctcggtaacgaccgcgtttaccgagagcaggactctcgacacagaaatacactcgacaaagaaatctttgccgagagccaaacactcggcgaacggcagcgctcggcaaagggtcgtcagccgccgtctaaagctgacggtcgttatctttgtcgagtgccccctcgtccgacactcagtagagcaagcttgccgagtgccatccttggacactcgataaagtatattttatttttttttattttgccaaccaaactttttgtggtatgttcctacactatgtagatctacatgtaccattttggcacaattacaaaaatgttttctataactattagatttagttcgtttatttgaatttcttcggaaaattcacatatgaactgcaagtcactcgaaacatgaaaaaccgtgcatgcaaaataaatgatatgcatgttatctagcacaagttacgaccgaattcagaagcagaccagaatcttcaagcaccatgctcactaaacatgaccgtgaacttgttatccagttgtttaaaaattgtataaaacacaaataaagtcagaaattaatgaaacttgtccacatgtcatgatatcatatatagaggttgtgataaaaatttgataatgtttcggtaaagttgtgacgtactatgtgtagaaacctaagtgacctacacataaaatcatagagtttcaatgtagttcactcgacaaagactttgtcaagtgtccgataaaaagtattcagcaaagaagccgttgtcgatttactgttcgtcgagatctctttgccgagtgtcacactaggcaaagtctttacggagtgtttttcaggctttgacactcggcaaagcgctcgattccagtagtgacagtaatttgcatcaaaaatagccgagagatttaaaatgagtcaactaatagaccaactaattattagctattagtcgttagcttctttaatctaagctaaaaccaactaatagcttatttgttgaattacaattagctcaacggaattctctgttttttctataaaaaaaagggaaactgcccctcatttacagcaaactgtccgctgcctgtcgtccagatacaatgaacgtacctagtaggaactcttttacacgctcggtcgctcgccgcggatcggagtcccaggaacacgacaccactgtggaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgccacctccgctttagttctgcatacagccaacccaacacacacccgagcatatcacagtgacagacactaca

SEQ ID NO: 12 - DNA, 314 bp, synthetic construct
  source 1..314: mol_type = other DNA
ctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaat